(19) United States
(12) Patent Application Publication
(10) Pub. No.: US 2009/0082203 A1
(43) Pub. Date: Mar. 26, 2009

(54) INSECTICIDAL BIS(SUBSTITUTED PHENYL)-1-{[4-(SATURATED HETEROCYCLYL-SUBSTITUTED)PHENYLMETHYL]- (4-PIPERIDYL)}METHANE DERIVATIVES

(75) Inventors: John W. Lyga, Basking Ridge, NJ (US); David S. Rosen, Princeton, NJ (US); Y. Larry Zhang, Kendall Park, NJ (US); Ping Ding, Lawrenceville, NJ (US); Robert N. Henrie, II, Pennington, NJ (US); Frank J. Zawacki, Yardley, PA (US); Charles E. Hatch, III, Pennington, NJ (US); Shunxiang Zhang, Plainsboro, NJ (US); Jaidev S. Goudar, West Windsor, NJ (US)

Correspondence Address: STERNE, KESSLER, GOLDSTEIN & FOX P.L.L.C., 1100 NEW YORK AVENUE, N.W., WASHINGTON, DC, 20005, US

(73) Assignee: Bayer Cropscience AG, Monheim (DE)

(21) Appl. No.: 11/721,661

(22) PCT Filed: Dec. 9, 2005
(86) PCT No.: PCT/US2005/044666

§ 371 Date: Mar. 28, 2008

Related U.S. Application Data
(60) Provisional application No. 60/635,545, filed on Dec. 13, 2004.

(51) Int. Cl. A01N 43/40; C07D 211/00; A01P 15/00

(52) U.S. Cl. 504/101; 546/184; 514/315; 504/130

(57) ABSTRACT

It has now been found that certain novel bis(substituted phenyl)-1 {[4-(saturated heterocyclyl-substituted)phenyl-methyl](4-piperidyl)}methane derivatives have provided unexpected insecticidal activity. These compounds are represented by formula (I); where R through R¹⁵, m, n, s, A, B, D and W are defined herein. In addition, compositions comprising an insecticidally effective amount of at least one compound of formula (I), and optionally, an effective amount of at least one of a second compound, with at least one insecticidally compatible carrier are also disclosed; along with methods of controlling insects comprising applying said compositions to a locus where insects are present or are expected to be present.

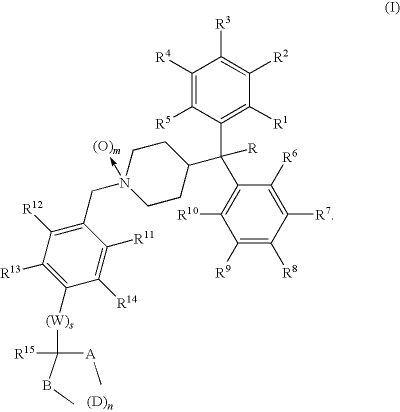

This application claims the benefit of U.S. Provisional Application No. 60/635,545, filed Dec. 13, 2004.

### FIELD OF THE INVENTION

The present invention generally relates to insecticidal compounds and their use in controlling insects. In particular, it pertains to insecticidal bis(substituted phenyl)-1-{[4-(saturated heterocyclyl-substituted)phenylmethyl] (4-piperidyl)}methane derivatives, N-oxides, and agriculturally acceptable salts thereof, compositions of these insecticides, and methods for their use in controlling insects.

### BACKGROUND OF THE INVENTION

It is well known that insects in general can cause significant damage, not only to crops grown in agriculture, but also, for example, to structures and turf where the damage is caused by soil-borne insects, such as termites and white grubs. Such damage may result in the loss of millions of dollars of value associated with a given crop, turf or structures. Thus, there is a continuing demand for new insecticides that are safer, more effective, and less costly. Insecticides are useful for controlling insects which may otherwise cause significant damage to crops such as wheat, corn, soybeans, potatoes, and cotton to name a few. For crop protection, insecticides are desired which can control the insects without damaging the crops, and which have no deleterious effects to mammals and other living organisms.

A number of patents disclose a variety of insecticidally active substituted piperidine and piperazine derivatives. For example, as set forth in U.S. Pat. No. 5,569,664, compounds of the following structure are reported to be insecticidally active:

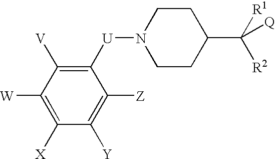

where U is selected from —(CH₂)ₙ— and ethylidine, where n is 1, 2, or 3; Q is selected from hydrogen, hydroxy, sulfhydryl, and fluorine; V is selected from hydrogen, halogen, alkyl, haloalkyl, alkoxy, alkylthio, alkylsulfinyl, alkylsilyloxy, dialkylamino, cyano, nitro, hydroxy, and phenyl; W is selected from hydrogen, halogen, alkyl, haloalkyl, alkoxy, nitro, amino, phenoxy, and phenylalkoxy; X is selected from hydrogen, hydroxy, halogen, alkyl, alkoxyalkyl, alkoxy, cycloalkylalkoxy, haloalkoxy, alkenyloxy, alkynyloxy, alkylsilyloxy, alkylthio, haloalkylthio, cyano, cyanoalkoxy, nitro, amino, monoalkylamino, dialkylamino, alkylaminoalkoxy, alkylcarbonylamino, alkoxycarbonylamino, alkylcarbonyl, alkoxycarbonyl, alkylaminocarbonyl, aminocarbonyloxy, phenyl, phenylalkoxy, phenoxy, and phenoxyalkyl; Y and Z are independently selected from hydrogen and alkoxy; R¹ and R² are independently selected from phenyl substituted with halogen, alkyl, haloalkyl, haloalkoxy, alkoxyalkyl, hydroxy, arylthio, alkoxy, dialkylamino, dialkylaminosulfonyl, hydroxyalkylaminocarbonyl, alkylsulfonyloxy, and haloalkylsulfonyloxy; and the corresponding N-oxides and agriculturally acceptable salts.

As set forth in U.S. Pat. No. 5,639,763 compounds of the following structure are reported to be insecticidally active:

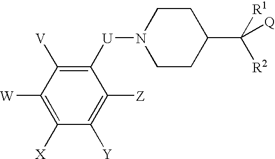

where U is selected from —(CH₂)ₙ— and ethylidine, where n is 1, 2, or 3; Q is selected from hydrogen, hydroxy, sulfhydryl, and fluorine; V is selected from hydrogen, halogen, alkyl, haloalkyl, alkoxy, alkylthio, alkylsulfinyl, alkylsilyloxy, dialkylamino, cyano, nitro, hydroxy, and phenyl; Y and Z are independently selected from hydrogen and alkoxy; W and X taken together is —OCH₂CH₂O—, —CH₂C(CH₃)₂O—, —OC(CH₃)₂O—, or —N═C(C₂H₅)O—; R¹ and R² are independently selected from phenyl substituted with halogen, alkyl, haloalkyl, haloalkoxy, alkoxyalkyl, hydroxy, arylthio, alkoxy, dialkylamino, dialkylaminosulfonyl, hydroxyalkylaminocarbonyl, alkylsulfonyloxy, and haloalkylsulfonyloxy; and the corresponding N-oxides and agriculturally acceptable salts.

As set forth in U.S. Pat. No. 5,795,901 compounds of the following structure are reported to be insecticidally active:

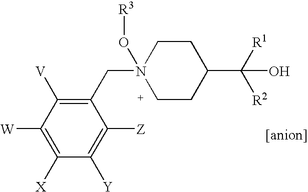

where V, W, Y, and Z are hydrogen; X is alkoxy, cycloalkoxy, alkoxycarbonyl, alkoxycarbonylamino, or a five- or six-membered heteroaryl or heteroaryloxy, each heteroaryl optionally substituted with halogen, cyano, alkyl, haloalkyl, alkoxy, haloalkoxy, alkoxyalkyl, or haloalkoxyalkyl; R¹ and R² are independently selected from haloalkyl, phenyl substituted with halogen, halothio, haloalkyl, or haloalkoxy; or a five- or six-membered heteroaryl substituted with halogen or alkyl; R³ is alkyl, haloalkyl, hydroxyalkyl, alkoxyalkyl, dialkylaminoalkyl, alkylaminocarbonyloxyalkyl, alkylthioalkyl, alkylsulfonylalkyl, alkylcarbonyloxyalkyl, alkoxycarbonylalkyl, carboxyalkyl, carboxyarylalkyl, arylcarbonyl, sulfonato, or sulfonatoalkyl, and may bear a negative charge resulting in an inner salt, and a separate anion is chloride, bromide, iodide, or a phenyl, or alkyl sulfate or sulfonate.

As set forth in U.S. Pat. No. 5,939,438 compounds of the following structure are reported to be insecticidally active:

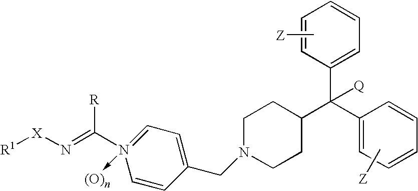

where R is hydrogen, halogen, alkyl, alkoxy, or dialkylamino; R¹ is hydrogen, alkyl, haloalkyl, alkoxyalkyl, alkylcarbonyl, or alkylaminocarbonyl; Q is fluoro or hydroxy; X is oxygen or NR²; Z is halogen, haloalkyl, haloalkoxy, pentahalothio, haloalkylthio, haloalkylsulfinyl, haloalkylsulfonyl, or —OCF₂O— attached to two adjacent carbon atoms of the phenyl ring; n is 0 or 1; and, when X is NR², R² is hydrogen, alkyl, alkylcarbonyl, alkoxycarbonyl, or R¹ and R² taken together may be —CₘH₂ₘ—, or —C₂H₄OC₂H₄—, where m is 3-9; and their agriculturally acceptable salts.

As set forth in U.S. Pat. No. 6,017,931 compounds of the following structure are reported to be insecticidally active:

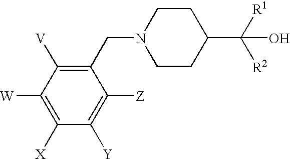

where V, W, and Z are hydrogen; X is selected from alkoxy, haloalkoxy, alkoxyalkyl, cycloalkylalkoxyl, halocycloalkylalkoxy, alkoxycarbonyl, haloalkoxycarbonyl, cycloalkylalkoxylcarbonyl, halocycloalkylalkoxylcarbonyl, alkoxyalkoxycarbonyl, alkoxycarbonylamino, haloalkoxycarbonylamino, cycloalkylalkoxycarbonylamino, halocycloalkylalkoxycarbonylamino, alkylaminocarbonyl, haloalkylaminocarbonyl, cyanoalkoxycarbonylamino, phenylcarbonylamino, and phenoxycarbonyl, each cycloalkyl moiety or phenyl ring optionally substituted with halogen; Y is selected from hydrogen or halogen; R¹ and R² are independently selected from phenyl or pyridyl, each substituted with haloalkyl, haloalkoxy, or alkylthio, and the corresponding N-oxides and agriculturally acceptable salts.

As set forth in U.S. Pat. No. 6,030,987 compounds of the following structure are reported to be insecticidally active:

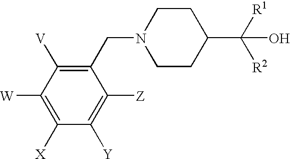

where V, W, Y and Z are hydrogen; X is a five- or six-membered heterocycle optionally substituted with halogen, alkyl, alkoxy, alkoxyalkyl, cyano, aminocarbonyl, haloalkyl, haloalkoxy, or haloalkoxyalkyl; and the heterocycle is optionally connected to the phenyl ring through a —O—, —S—, —(CH₂)ₚ—, —C(O)—, or —O(CR³R⁴)_{q}— linkage; R¹ and R² are independently selected from phenyl or pyridyl, each substituted with haloalkyl, or haloalkoxy; R³ and R⁴ are independently selected from hydrogen and methyl; n and p are independently 1, 2, or 3; and q is 1 or 2, and the corresponding N-oxides and agriculturally acceptable salts.

As set forth in U.S. Pat. No. 6,184,234 compounds of the following structure are reported to be insecticidally active:

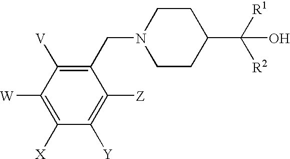

where V, W, Y and Z are hydrogen; X is a five- or six-membered heterocycle optionally substituted with bromine, chlorine, fluorine, alkyl, alkoxy, alkoxyalkyl, cyano, aminocarbonyl, haloalkyl, haloalkoxy, or haloalkoxyalkyl; and the heterocycle is optionally connected to the phenyl ring through a —O—, —S—, —(CH₂)ₚ—, —C(O)—, or —O(CR³R⁴)_{q}— linkage; R¹ and R² are independently selected from i) phenyl or pyridyl, each substituted with pentahalothio, haloalkylthio, haloalkylsulfinyl, or haloalkylsulfonyl; ii) phenyl substituted with —OC(M)₂O—, where M is bromine, chlorine, or fluorine to provide a dihalobenzodioxolyl fused ring; or iii) pyridyl substituted with —OC(M)₂O—, to provide a dihalodioxoleneopyridyl fused ring; R³ and R⁴ are independently selected from hydrogen and methyl; n and p are independently 1, 2, or 3; and q is 1 or 2, and the corresponding N-oxides and agriculturally acceptable salts.

As set forth in United States Statutory Invention Registration H1,838 compounds of the following structure are reported to be insecticidally active:

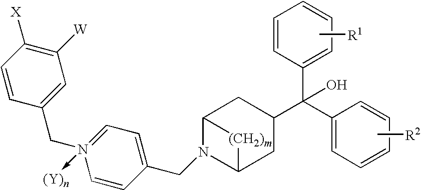

where m is 2 or 3; n is 0 or 1; X is hydrogen, alkoxy, cycloalkylalkoxy, haloalkoxyimino, or a five- or six-membered heteroaryl or heteroaryloxy in which one or more hetero atoms may be optionally substituted with alkyl; R¹ and R² are independently selected from hydrogen, haloalkyl, halothio, or haloalkoxy; and when n is 1, Y represents (a) an N-oxide of the ring nitrogen; or (b) an agriculturally acceptable anionic salt of the ring nitrogen; or (c) forms an OR³ linkage in which R³ is selected from hydrogen, alkyl, alkoxycarbonylalkyl, hydroxycarbonylethyl in association with an agriculturally acceptable anion resulting in an ionic salt, or R³ is an oxycarbonylalkyl group bearing a negative charge resulting in an inner salt.

As set forth in United States Statutory Invention Registration H1,996 photostable, agriculturally acceptable acid salts of an organic or inorganic acid of the following structure are reported to be insecticidally active:

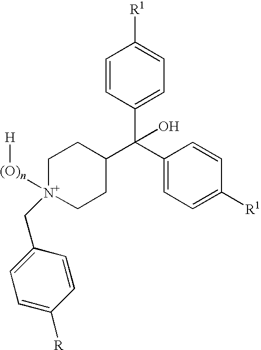

where R is alkoxycarbonyl, alkoxycarbonylamino, cycloalkylalkoxy, 2-alkyl-2H-tetrazol-5-yl, or 2-haloalkyl-2H-tetrazol-5-yl; R¹ is trihaloalkyl, or trihaloalkoxy; n is 0, or 1; and said salt is at least 2.5 times more photostable than its non-ionic parent and is derived from hydrochloric acid, hydrobromic acid, boric acid, phosphoric acid, maleic acid, fumaric acid, phthalic acid, D-glucuronic acid; the sulfonic acid R²SO₃H where R² is alkyl, haloalkyl, hydroxyalkyl, D-10-camphoryl, or phenyl optionally substituted with alkyl or halogen; the carboxylic acid R³CO₂H where R³ is hydrogen, alkyl, trihaloalkyl, carboxyl, phenyl optionally substituted with alkyl or halogen, or pyridyl; the boronic acid R⁴B(OH)₂ where R⁴ is alkyl or phenyl optionally substituted with alkyl or halogen; the phosphonic acid R⁵PO₃H₂ where R⁵ is alkyl, haloalkenyl, or phenyl optionally substituted with alkyl or halogen; the sulfuric acid R⁶⁰SO₃H where R⁶ is hydrogen or alkyl; or the alkanoic acid X—(CH₂)_{q}CO₂H where q is 0 to 11, X is halogen, trihaloalkyl, haloalkenyl, cyano, aminocarbonyl, or CO₂R⁷ where R⁷ is hydrogen or alkyl.

As set forth in United States Statutory Invention Registration H2,007 compounds of the following structures are reported to be insecticidally active:

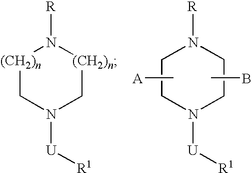

where A and B are independently selected from lower alkyl; U is selected from lower alkylidene, lower alkenylidene, and CH-Z, where Z is selected from hydrogen, lower alkyl, lower cycloalkyl, or phenyl; R is —CHR³R⁴ where R³ and R⁴ are independently selected from phenyl, optionally substituted with halogen, lower alkyl, lower haloalkyl, lower alkoxy, lower haloalkoxy, lower alkenyl, or phenyl; R¹ is phenyl, naphthyl, tetrazolylphenyl, phenylcyclopropyl, phenoxyphenyl, benzyloxyphenyl, pyridylphenyl, pyridyloxyphenyl, or thiadiazolyloxyphenyl, each optionally substituted with halogen, cyano, hydroxy, lower alkyl, lower haloalkyl, lower alkoxy, amino, lower dialkylamino, nitro, lower haloalkylsulfonyloxy, lower alkylcarbonyloxy, lower alkylcarbonylamino, lower alkoxycarbonyl, lower alkoxyalkoxycarbonyl, lower cycloalkylalkoxycarbonyl, lower alkoxyalkylalkoxycarbonyl, lower alkoxycarbonylamino, alkoxythiocarbonylamino, lower alkyldithiocarbonylamino, lower dialkyldioxolylalkoxycarbonylamino, or halophenylamino; or lower alkyl substituted with any one of the foregoing cyclic R¹ groups; m is 2 or 3; and n is 1, 2, or 3.

As set forth in unexamined Japanese Patent Application 2002-220372 compounds of the following structures are reported to be insecticidally active:

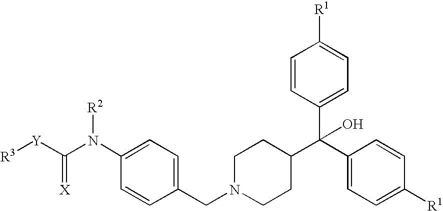

where R¹ and R² are independently selected from hydrogen, halogen, lower alkyl, lower haloalkyl, lower alkoxy, lower haloalkoxy, or lower alkylsulfonyloxy; R² is selected from hydrogen, lower alkyl, lower alkenyl, lower alkoxyalkyl, or lower alkylcarbonyl; X and Y are independently oxygen or sulfur; R³ is selected from lower alkenyl, or lower alkynyl, which are optionally substituted with hydroxy, halogen, lower alkoxy, lower haloalkoxy, lower alkylthio, lower alkylsulfinyl, lower alkylsulfonyl, lower cycloalkyl, lower alkoxyalkoxy, amino, lower alkylamino, lower dialkylamino, lower alkoxycarbonyl, nitro, cyano, trimethylsilyl, phenyl, or lower cycloalkenyl; and the corresponding N-oxides and salts.

As set forth in PCT Publication WO 02/068392A1 compounds of the following structures are reported to be insecticidally active:

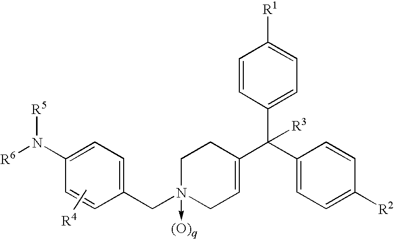

where R¹ and R² are independently selected from halogen, C₁-C₆alkyl, haloC₁-C₆alkyl, C₁-C₆alkoxy, haloC₁-C₆alkoxy, —S(═O)ₚ—R⁹, or SF₅; R³ is hydrogen, hydroxy, C₁-C₆alkoxy, or —OC(═O)—C₁-C₆alkyl; R⁴ is hydrogen, halogen, C₁-C₆alkyl, haloC₁-C₆alkyl, C₁-C₆alkoxy, haloC₁-C₆alkoxy, or —S(═O)ₚ—R⁹, or —SCN; R⁵ and R⁶ are independently selected from C₁-C₁₂alkyl, haloC₁-C₁₋₂alkyl, C₂-C₁₂alkenyl, haloC₂-C₁₂alkenyl, C₂-C₁₂alkynyl, haloC₂-C₁₂alkynyl, C₃-C₈cycloalkyl, —C(═O)—OR⁷, —C(═S)—OR⁸, —C(═Y)-ZR⁸, —S(═O)ₚ—R⁹, aryl, arylC₁-C₆alkyl, heterocycle, heterocycleC₁-C₆alkyl, each substituted in the ring from one to five times independently of one another by halogen, hydroxy, cyano, nitro, C₁-C₆alkyl, haloC₁-C₆alkyl, C₁-C₆alkoxy, haloC₁-C₆alkoxy; or in common together with the nitrogen atom to which they are attached to form a heterocyclic ring which is substituted or unsubstituted; Y is oxygen or sulfur; X is a bond, —NR¹⁰—, or sulfur; R⁷ is C₁-C₆alkoxy-C₁-C₆alkyl, C₁-C₆alkylthio-C₁-C₆alkyl, C₁-C₆alkylamino-C₁-C₆alkyl, C₃-C₆alkynyl, C₁-C₆alkyl-S(═O)ₚ—C₁-C₆alkyl, C₃-C₈cycloalkyl, aryl, aryl-C₁-C₆alkyl, heterocyclyl, or heterocyclyl-C₁-C₆alkyl each substituted in the ring from one to five times independently of one another by halogen, cyano, nitro, C₁-C₆alkyl, haloC₁-C₆alkyl, C₁-C₆alkoxy, or haloC₁-C₆alkoxy; R⁸ is C₁-C₆alkyl, haloC₁-C₆alkyl, C₁-C₆alkoxy-C₁-C₆alkyl, C₁-C₆alkylthio-C₁-C₆alkyl, C₂-C₆alkenyl, C₃-C₆alkynyl, C₁-C₆alkyl-S(═O)ₚ—C₁-C₆alkyl, C₃-C₈cycloalkyl, aryl, aryl-C₁-C₆alkyl, heterocyclyl, or heterocyclyl-C₁-C₆alkyl, or is C₃-C₈cycloalkyl, aryl, aryl-C₁-C₆alkyl, heterocyclyl, or heterocyclyl-C₁-C₆alkyl each substituted in the ring from one to five times independently of one another by halogen, cyano, nitro, C₁-C₆alkyl, haloC₁-C₆alkyl, C₁-C₆alkoxy, or haloC₁-C₆alkoxy; R⁹ is C₁-C₆alkyl, C₃-C₈cycloalkyl, haloC₁-C₆alkyl, or benzyl; R¹⁰ is hydrogen, C₁-C₆alkyl, C₃-C₈cycloalkyl, haloC₁-C₆alkyl, or benzyl; p is 0, 1, or 2; q is 0 or 1; and, where appropriate, E/Z isomers, E/Z isomer mixtures and/or toutomers, each in free form or in salt form.

As set forth in German Offenlegungsschrift DE 10 2004 010 086 A1, compounds of the following structure are reported to be insecticidally active:

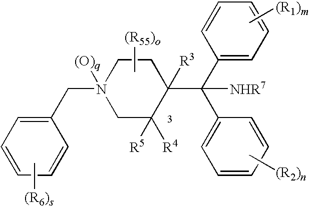

where R₁ and R₂ are selected from hydrogen, halogen, (C₁-C₁₂)alkyl, cycloalkyl, haloalkyl, halocycloalkyl, Q, alkoxy, haloalkoxy, alkenyloxy, alkynyloxy, haloalkenyloxy, haloalkynyloxy, SF₅, CON(R₁₁)₂, OCON(R₁₁)₂, CN, NO₂, SO₂N(R₁₁)₂, S(O)ₚ-alkyl, S(O)ₚ-haloalkyl, OS(O)ₚ-alkyl or OS(O)ₚ-haloalkyl; Q is (C₂-C₄)alkenyl, (C₂-C₄)alkynyl, (C₂-C₄)haloalkenyl or (C₂-C₄)haloalkynyl; R₃ and R₄ are hydrogen or together form a bond; R₅ is hydrogen, alkyl, haloalkyl, cycloalkyl, (C₂-C₄)alkenyl, (C₂-C₄)alkynyl, alkoxy, alkoxyalkyl, haloalkoxy, alkenyloxy, alkynyloxy, alkylthio, alkylsulfinyl, or alkylsulfonyl; R₅₅ is hydrogen, alkyl, haloalkyl, cycloalkyl, (C₂-C₄)alkenyl, (C₂-C₄)alkynyl, alkoxy, alkoxyalkyl, haloalkoxy, alkenyloxy or alkynyloxy, where o is 1-3; R₆ is hydrogen, halogen, CN, NO₂, alkyl, haloalkyl, cycloalkyl, halocycloalkyl, cycloalkoxy, alkoxy, haloalkoxy, Q, alkenyloxy, alkynyloxy, haloalkenyloxy, haloalkynyloxy, alkylcarbonyl, haloalkylcarbonyl, alkoxycarbonyl, haloalkoxycarbonyl, N(R₁)₂, CON(R₁₁)₂, OCON(R₁₁)₂, SO₂N(R₁₁)₂, S(O)ₚ-alkyl, S(O)ₚ-haloalkyl, OS(O)ₚ-alkyl, OS(O)ₚ-haloalkyl, N(R₁₆)—C(Y)-ZR₇ or C(R₁₃)═N═W—R₁₄, or aryl, benzyl, aryloxy, benzyloxy, heterocyclyl or heterocyclyloxy (all optionally substituted with 1-5 of halogen, CN, NO₂, alkyl, (C₃-C₈)cycloalkyl, (C₃-C₈)cycloalkylalkyl, haloalkyl, alkoxy, cycloalkoxy, (C₃-C₈)cycloalkoxyalkyl, (C₃-C₈)cycloalkylalkoxy haloalkoxy, Q, alkenyloxy, alkynyloxy, haloalkenyloxy, haloalkynyloxy, phenyl, benzyl, phenoxy, benzyloxy, heterocycle or heterocycloxy); R₇ is hydrogen, (C₁-C₁₂)alkyl, (C₃-C₈)cycloalkyl, haloalkyl, halocycloalkyl, Q, COR₈, OR₉, NHR₉, N═R(R₁₀)₂, phenyl or benzyl; R₈ is hydrogen, (C₁-C₁₂)alkyl, (C₃-C₈)cycloalkyl, haloalkyl, halocycloalkyl, Q, OR₉ or NHR₉; R⁹ and R¹⁰ are alkyl, haloalkyl, halocycloalkyl, alkenyl, alkynyl, haloalkenyl, haloalkynyl, (C₃-C₈)cycloalkyl, phenyl or benzyl; R¹¹ is hydrogen, (C₁-C₁₂)alkyl, (C₁-C₁₂)haloalkyl, (C₂-C₁₂)alkenyl, (C₂-C₁₂)alkynyl, (C₂-C₁₂)haloalkenyl, (C₂-C₁₂)haloalkynyl, CONR₁₄R₁₅, CSNR₁₄R₁₅ or S(O)ₚ—R₁₄; or (C₃-C₈)cycloalkyl, aryl, arylalkyl, aryloxy, heterocyclyl, or heterocyclyloxy (all optionally substituted in the ring by 1-5 of halogen, OH, CN, NO₂, alkyl, haloalkyl, alkoxy or haloalkoxy), or N(R₁₁)₂ optionally substituted with heterocycle; R₁₂ is hydrogen, alkyl or benzyl; R₁₃ is halogen, alkyl, (C₃-C₈)cycloalkyl, (C₃-C₈)cycloalkylalkyl, haloalkyl, alkoxy, (C₃-C₈)cycloalkoxy, (C₃-C₈)cycloalkoxyalkyl, haloalkoxy, mono- or dialkylamino; R₁₄ and R₁₅ are hydrogen, alkyl, (C₃-C₈)cycloalkyl, (C₃-C₈)cycloalkylalkyl, haloalkyl, Q or alkylcarbonyl; R₁₆ is hydrogen, alkyl, alkoxyalkyl, cycloalkyl, haloalkyl, alkenyl or alkynyl; R₁₇ is hydrogen, alkyl, haloalkyl, alkoxyalkyl, (C₃-C₈)cycloalkyl, (C₃-C₈)halocycloalkyl, alkenyl, alkynyl, haloalkenyl, haloalkynyl, aryl, arylalkyl or heterocyclyl (all optionally substituted by 1-3 of halogen, CN, NO₂, alkyl, (C₃-C₈)cycloalkyl, haloalkyl, alkoxy, haloalkoxy, Q, alkenyloxy, or alkynyloxy); m, n and s are 1-5; o is 1-3; p is 0-2; q is 0 or 1; Y is O or S; Z is a direct bond, O, S or NR₁₈; R₁₈ is hydrogen, alkyl, alkoxyalkyl, (C₃-C₈)cycloalkyl, haloalkyl, alkenyl or alkynyl; and W is O, NH or N(alkyl).

There is no disclosure or suggestion in any of the citations set forth above of the bis(substituted phenyl)-1-{[4-(saturated heterocyclyl-substituted)phenylmethyl](4-piperidyl)}methane derivatives or the surprising insecticidal activity thereof described in the present invention.

### SUMMARY OF THE INVENTION

In accordance with the present invention, it has now been found that certain bis(substituted phenyl)-1-{[4-(saturated heterocyclyl-substituted)phenylmethyl](4-piperidyl)}methane derivatives, (hereinafter termed “compounds of formula I”), N-oxides, and agriculturally acceptable salts thereof are surprisingly insecticidally active when used in the insecticidal compositions and methods of this invention. The compounds of formula I are represented by the following general formula I:

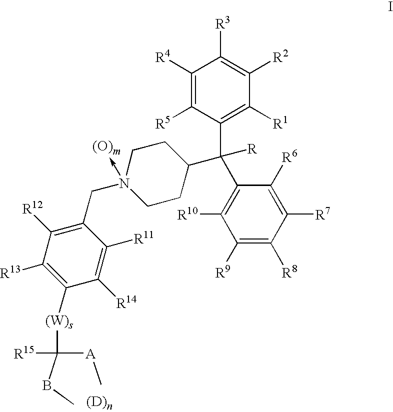

wherein
R is selected from hydrogen, OR¹⁶, SR¹⁶, halogen, cyano, alkyl, haloalkyl, alkylsilyl, alkoxysilyl, NR¹⁶R¹⁷, C(═O)R¹⁶, NHC(═O)R¹⁶, NHC(═O)OR¹⁶, NHC(═O)NHR¹⁶ and NHC(═S)NHR¹⁶; where R¹⁶ and R¹⁷ are independently selected from hydrogen, alkyl, haloalkyl, hydroxyalkyl, alkoxyalkyl, alkylsilyl, alkoxysilyl, aryl, arylalkyl and heteroaryl;
R¹ through R¹⁰, inclusively, are independently selected from hydrogen, halogen, alkyl, haloalkyl, alkoxy, haloalkoxy, S(O)ₚ-alkyl, S(O)ₚ-haloalkyl, pentahalothio and nitro; where p is an integer selected from 0, 1 or 2;
   and
   R¹ and R², R² and R³, R⁶ and R⁷, and R⁷ and R⁸ may be taken together with —OC(halogen)₂O— to form benzo-fused rings;
      m is an integer selected from 0 or 1;
      s is an integer selected from 0 or 1;
W is selected from —CR³⁰R³¹— or —OCR³OR³—; where R³⁰ and R³¹ are independently selected from hydrogen or alkyl;
R¹¹ through R¹⁴, inclusively, are independently selected from hydrogen, halogen, alkyl, haloalkyl, alkoxyalkyl, OR¹⁶, S(O)ₚR¹⁶, NR¹⁶R¹⁷ and aryl; where p, R¹⁶ and R¹⁷ are previously described;
R¹⁵ is selected from hydrogen, alkyl, cycloalkyl, alkenyl, alkynyl, haloalkyl, hydroxyalkyl, alkoxyalkyl, OR¹⁶, SR¹⁶, aryl, arylalkyl, aryloxyalkyl and heteroaryl; where R¹⁶ is previously described;
   n is an integer selected from 0 or 1;
   and,
   when n is 0 or 1;
A and B are independently selected from —O(C═O)—, —C(═O)O—, —O(CR¹⁸R¹⁹)_{q}—, —(CR¹⁸R¹⁹)_{q}O—, —S(O)ₚ(CR¹⁵R¹⁹)_{q}—, —(CR¹⁸R¹⁹)_{q}S(O)ₙ—, —(CR¹⁵R¹⁹)_{q}NR²⁰ and —NR²⁰(CR¹⁸R¹⁹)_{q}—, where p is previously described, and q is an integer selected from 1 to 7, provided that the sum of q is 8 or less; R¹⁸ and R¹⁹ are independently selected from hydrogen, alkyl, alkenyl, alkynyl, haloalkyl, hydroxyalkyl, alkoxyalkyl, alkenyloxyalkyl, aryl, aryloxyalkyl, arylalkoxycarbonylaminoalkyl, heterocyclalkyl and heteroaryl; and R²⁰ is selected from hydrogen, alkyl, haloalkyl, hydroxyalkyl, alkoxyalkyl, acyl, alkylcarbonyl, alkoxycarbonyl, aminocarbonyl, alkylaminocarbonyl, dialkylaminocarbonyl, aryl, arylalkyl, silyl and phosphoryl;
   and when n is 1;
D is selected from —O—, —S—, —NR²⁰—, —C═NOR²⁰—, —C(halogen)₂-, —CR²¹R²²—, —C(R²¹)(OR²²)—, —C(OR²¹)(OR²²)—, SiR²¹R²²—, —Si(OR²¹)(OR²²)—, P(═O)ᵣR²¹, P(═O)ᵣ(OR²¹), BR²¹, B(OR²¹), C(═O), C═CR²³R²⁴, —CR²³═CR²⁴— and

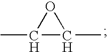

where R²⁰ is previously described; R²¹ and R²² are independently selected from hydrogen, alkyl, alkenyl, alkynyl, haloalkyl, hydroxyalkyl, alkoxyalkyl, amino, aryl and heteroaryl, and where R²¹ and R²² may be taken together to form a spiro ring; R²³ and R²⁴ are independently selected from hydrogen, halogen, alkyl, haloalkyl, hydroxyalkyl, alkoxyalkyl, alkylsilyl, alkoxysilyl, aryl, arylalkyl and heteroaryl; and r is an integer selected from 0 or 1;
or
when n is 1;
A and B are independently selected from selected from —O—, —S(O)ₚ—, —O(CR¹⁸R¹⁹)_{q}—, —(CR¹⁸R¹⁹)_{q}O—, —NR²⁰— and —(CR²¹R²²)ₜ—, where p, q, R¹⁸, R¹⁹, R²⁰, R²¹ and R²² are previously described; and t is an integer selected from 1, 2, 3, or 4;
   and,
   D is a phenylene moiety

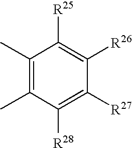

which,
when taken together with A and B, forms a benzo-fused ring, where R²⁵ through R²⁸ are independently selected from hydrogen, halogen, alkyl, haloalkyl, hydroxyalkyl, alkoxy, alkoxyalkoxy, alkylthio, thioalkyl, cyano, alkoxycarbonyl, aryl, aryloxy, heteroaryl and heteroaryloxy;
   or
   when n is 1;
A and B are independently selected from selected from —O—, —S(O)ₚ—, —O(CR¹⁸R¹⁹)_{q}—, —(CR¹⁸R¹⁹)_{q}O—, —NR²⁰— and —(CR²¹R²²)ₜ—, where p, q, R¹⁸, R¹⁹, R²⁰, R²¹ and R²² are previously described; and t is an integer selected from 1, 2, 3, or 4;
   and,
   D is a cyclic moiety selected from:

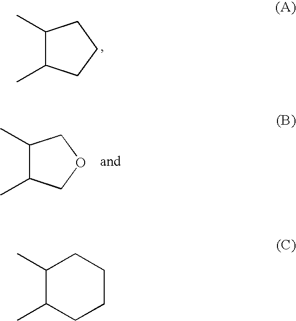

which,
when taken together with A and B, form a fused ring;
and agriculturally acceptable salts thereof;
provided that when R is OR¹⁶ where R¹⁶ is hydrogen; m is 0; R¹¹ through R¹⁵, inclusively are hydrogen; A and B are each —O(CR¹⁸R¹⁹)_{q}— where R¹⁸ and R¹⁹ are each hydrogen and q is 1; and n is 0, and i) when R¹, R², R⁴R⁵, R⁷, R⁸, R⁹ and R¹⁰ are hydrogen and R³ is trifluoromethoxy, then R⁶ is other than trifluoromethoxy; or ii) when R¹, R², R⁴R⁵, R⁶, R¹, R⁸ and R⁹ are hydrogen and R³ is trifluoromethoxy, then R¹⁰ is other than trifluoromethoxy; or iii) when R², R³, R⁴R⁵, R⁶, R⁷, R⁹ and R¹⁰ are hydrogen and R⁸ is trifluoromethoxy, then R¹ is other than trifluoromethoxy, or iv) when R¹, R², R³ R⁴, R⁵, R⁶, R⁷, R⁹ and R¹⁰ are hydrogen and R⁸ is trifluoromethoxy, then R⁵ is other than trifluoromethoxy.

One skilled in the art will, of course, recognize that within the description set forth above, when A and B are independently selected from —C(═O)O—, —(CR¹⁸R¹⁹)_{q}O— and —(CR¹⁸R¹⁹)_{q}S(O)ₚ—, then D is not selected from —O— or —S—.

The present invention is also directed to compositions containing an insecticidally effective amount of at least one of a compound of formula I, and optionally, an effective amount of at least one of a second compound, with at least one agriculturally acceptable extender or adjuvant.

The present invention is also directed to methods of controlling insects, where control is desired, which comprise applying an insecticidally effective amount of the above composition to the locus of crops, or other areas where insects are present or are expected to be present. Other aspects of the present invention will become apparent.

### DETAILED DESCRIPTION OF THE INVENTION

One aspect of the present invention relates to certain new and useful compounds, namely certain novel bis(substituted phenyl)-1-{[4-(saturated heterocyclyl-substituted)phenylmethyl](4-piperidyl)}methane derivatives as depicted in general formula I:

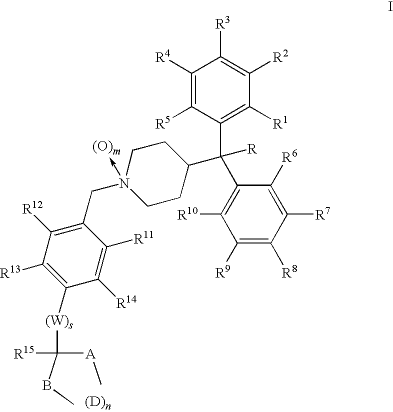

wherein
R is selected from hydrogen, OR¹⁶, SR¹⁶, halogen, cyano, alkyl, haloalkyl, alkylsilyl, alkoxysilyl, NR¹⁶R¹⁷, C(═O)R¹⁶, NHC(═O)R¹⁶, NHC(═O)OR¹⁶, NHC(═O)NHR¹⁶ and NHC(═S)NHR¹⁶; where R¹⁶ and R¹⁷ are independently selected from hydrogen, alkyl, haloalkyl, hydroxyalkyl, alkoxyalkyl, alkylsilyl, alkoxysilyl, aryl, arylalkyl and heteroaryl;
R¹ through R¹⁰, inclusively, are independently selected from hydrogen, halogen, alkyl, haloalkyl, alkoxy, haloalkoxy, S(O)ₚ-alkyl, S(O)ₚ-haloalkyl, pentahalothio and nitro; where p is an integer selected from 0, 1 or 2;
   and
   R¹ and R², R² and R³, R⁶ and R⁷, and R⁷ and R⁸ may be taken together with —OC(halogen)₂O— to form benzo-fused rings;
      m is an integer selected from 0 or 1;
      s is an integer selected from 0 or 1;
W is selected from —CR³⁰R³¹— or —OCR³⁰R³¹—; where R³⁰ and R³¹ are independently selected from hydrogen or alkyl;
R¹¹ through R¹⁴, inclusively, are independently selected from hydrogen, halogen, alkyl, haloalkyl, alkoxyalkyl, OR¹⁶, S(O)ₚR¹⁶, NR¹⁶R¹⁷ and aryl; where p, R¹⁶ and R¹⁷ are previously described;
R¹⁵ is selected from hydrogen, alkyl, cycloalkyl, alkenyl, alkynyl, haloalkyl, hydroxyalkyl, alkoxyalkyl, OR¹⁶, SR¹⁶, aryl, arylalkyl, aryloxyalkyl and heteroaryl; where R¹⁶ is previously described;
   n is an integer selected from 0 or 1;
   and,
   when n is 0 or 1;
A and B are independently selected from —O(C═O)—, —C(═O)O—, —O(CR¹⁸R¹⁹)_{q}—, —(CR¹⁸R¹⁹)_{q}O—, —S(O)ₚ(CR¹⁸R¹⁹)_{q}—, —(CR¹⁸R¹⁹)_{q}S(O)ₚ—, —(CR¹⁸R¹⁹)_{q}NR²⁰— and —NR²⁰(CR¹⁸R¹⁹)_{q}—, where p is previously described, and q is an integer selected from 1 to 7, provided that the sum of q is 8 or less; R¹⁸ and R¹⁹ are independently selected from hydrogen, alkyl, alkenyl, alkynyl, haloalkyl, hydroxyalkyl, alkoxyalkyl, alkenyloxyalkyl, aryl, aryloxyalkyl, arylalkoxycarbonylaminoalkyl, heterocyclalkyl and heteroaryl; and R²⁰ is selected from hydrogen, alkyl, haloalkyl, hydroxyalkyl, alkoxyalkyl, acyl, alkylcarbonyl, alkoxycarbonyl, aminocarbonyl, alkylaminocarbonyl, dialkylaminocarbonyl, aryl, arylalkyl, silyl and phosphoryl;
   and when n is 1;
   D is selected from —O—, —S—, —NR²⁰—, —C═NOR²⁰—, —C(halogen)₂-, —CR²¹R²²—, C(R²¹)(OR²²)—, C(OR²¹)(OR²²)—, SiR²¹R²²—, —Si(OR²¹)(OR²²)—, P(═O)ᵣR²¹, P(═O)ᵣ(OR²¹), BR²¹, B(OR²¹), C(═O), C═CR²³R²⁴, —CR²³═CR²⁴— and

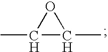

where R²⁰ is previously described; R²¹ and R²² are independently selected from hydrogen, alkyl, alkenyl, alkynyl, haloalkyl, hydroxyalkyl, alkoxyalkyl, amino, aryl and heteroaryl, and where R²¹ and R²² may be taken together to form a spiro ring; R²³ and R²⁴ are independently selected from hydrogen, halogen, alkyl, haloalkyl, hydroxyalkyl, alkoxyalkyl, alkylsilyl, alkoxysilyl, aryl, arylalkyl and heteroaryl; and r is an integer selected from 0 or 1;
or
when n is 1;
A and B are independently selected from selected from —O—, —S(O)ₚ—, —O(CR¹⁸R¹⁹)_{q}—, —(CR¹⁸R¹⁹)_{q}O—, —NR²⁰— and —(CR²¹R²²)ₜ—, where p, q, R¹⁸, R¹⁹, R²⁰R²¹ and R²² are previously described; and t is an integer selected from 1, 2, 3, or 4;
   and,
   D is a phenylene moiety

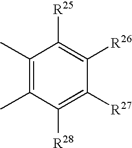

which,
when taken together with A and B, forms a benzo-fused ring, where R²⁵ through R²⁸ are independently selected from hydrogen, halogen, alkyl, haloalkyl, hydroxyalkyl, alkoxy, alkoxyalkoxy, alkylthio, thioalkyl, cyano, alkoxycarbonyl, aryl, aryloxy, heteroaryl and heteroaryloxy;
   or
   when n is 1;
A and B are independently selected from selected from —O—, —S(O)ₚ—, —O(CR¹⁸R¹⁹)_{q}—, —(CR¹⁸R¹⁹)_{q}O—, NR²⁰— and —(CR²¹, R²²)ₜ—, where p, q, R¹⁸, R¹⁹, R²⁰, R²¹ and R²² are previously described; and t is an integer selected from 1, 2, 3, or 4;
   and,
   D is a cyclic moiety selected from:

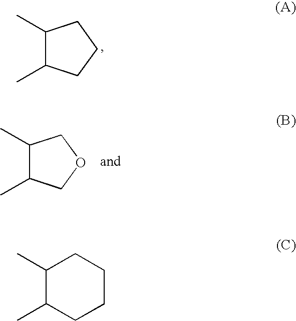

which,
when taken together with A and B, form a fused ring;
and agriculturally acceptable salts thereof;
provided that when R is OR¹⁶ where R¹⁶ is hydrogen; m is 0; R¹¹ through R¹⁵, inclusively are hydrogen; A and B are each —O(CR¹⁵R¹⁹)_{q}— where R¹⁸ and R¹⁹ are each hydrogen and q is 1; and n is 0, and i) when R¹, R², R⁴R⁵, R⁷, R⁸, R⁹ and R¹⁰ are hydrogen and R³ is trifluoromethoxy, then R⁶ is other than trifluoromethoxy; or ii) when R¹, R², R⁴R⁵, R⁶, R⁷, R⁸ and R⁹ are hydrogen and R³ is trifluoromethoxy, then R¹⁰ is other than trifluoromethoxy; or iii) when R², R³, R⁴R⁵, R⁶, R⁷, R⁹ and R¹⁰ are hydrogen and R⁵ is trifluoromethoxy, then R¹ is other than trifluoromethoxy, or iv) when R¹, R², R³R⁴, R⁶, R⁷, R⁹ and R¹⁰ are hydrogen and R⁸ is trifluoromethoxy, then R⁵ is other than trifluoromethoxy.

One skilled in the art will, of course, recognize that within the description set forth above, when A and B are independently selected from —C(═O)O—, —(CR¹⁸R¹⁹)_{q}O— and —(CR¹⁵R¹⁹)_{q}S(O)ₚ—, then D is not selected from —O— or —S—.

Preferred species are those compounds of formula I where R is selected from hydrogen, fluorine, OR¹⁶ and NR¹⁶R¹⁷, where R¹⁶ and R¹⁷ are hydrogen; i) R¹, R², R⁴, R⁵, R⁶, R⁷, R⁹ and R¹⁰ are each hydrogen, and R³ and R⁸ are each trifluoromethyl or trifluoromethoxy; or ii) R¹, R⁴, R⁵, R⁶, R⁹ and R¹⁰ are each hydrogen, and R², R³, R⁷ and R⁸ are independently selected from fluorine or chlorine; R¹¹ through R¹⁴, inclusively, are independently selected from hydrogen, halogen, alkyl and alkoxy; R¹⁵ is hydrogen or alkyl; n is 0; A and B are independently selected from —O(CR¹⁸R¹⁹)_{q}—, S(O)ₚ(CR¹⁸R¹⁹)_{q} and —NR²⁰(CR¹⁸R¹⁹)_{q}—, where p is 0, and q is an integer selected from 1 to 3.

Particularly preferred compounds of formula I are those where R is OR¹⁶ and R¹⁶ is hydrogen; R¹R², R⁴R⁵R⁶, R⁷, R⁹ and R¹⁰ are each hydrogen, and R³ and R⁸ are each trifluoromethyl or trifluoromethoxy; R¹¹ through R¹³, inclusively, are each hydrogen; R¹⁴ is hydrogen, halogen or alkyl; q is an integer selected from 1 to 2; R¹⁵ is hydrogen; and R¹⁸ and R¹⁹ are hydrogen or alkyl.

In certain cases the compounds within the scope of formula I may possess asymmetric centers, which can give rise to optical enantiomorphs and diastereomers. Compounds within the scope of formula I may exist in two or more forms, i.e., polymorphs, which are significantly different in physical and chemical properties. Compounds within the scope of formula I may also exist as tautomers, which are in equilibrium. Compounds within the scope of formula I may also possess acidic or basic moieties, which may allow for the formation of agriculturally acceptable salts or agriculturally acceptable metal complexes.

This invention includes the use of such enantiomorphs, polymorphs, tautomers, salts and metal complexes. Agriculturally acceptable salts and metal complexes include, without limitation, for example, ammonium salts, the salts of organic and inorganic acids, such as hydrochloric acid, sulfonic acid, ethanesulfonic acid, trifluoroacetic acid, methylbenzenesulfonic acid, phosphoric acid, gluconic acid, pamoic acid, and other acid salts, and the alkali metal and alkaline earth metal complexes with, for example, sodium, potassium, lithium, magnesium, calcium, and other metals.

The methods of the present invention are predicated on causing an insecticidally effective amount of a compound of formula I to be present within insects in order to kill or control the insects. Preferred insecticidally effective amounts are those that are sufficient to kill the insect. It is within the scope of the present invention to cause a compound of formula I to be present within insects by contacting the insects with a derivative of that compound, which derivative is converted within the insect to a compound of formula I. This invention includes the use of such compounds, which can be referred to as pro-insecticides.

Another aspect of the present invention relates to compositions containing an insecticidally effective amount of at least one compound of formula I, and, optionally, an effective amount of at least one additional compound, with at least one agriculturally acceptable extender or adjuvant.

Another aspect of the present invention relates to methods of controlling insects by applying an insecticidally effective amount of a composition set forth above to a locus of crops such as, without limitation, cereals, cotton, vegetables, and fruits, other areas where insects are present or are expected to be present, or adjacent to areas where insects are present or are expected to be present.

The present invention also includes the use of the compounds and compositions set forth herein for control of non-agricultural insect species, for example, ants, dry wood termites and subterranean termites as well as other insects; and also for use as pharmaceutical agents and compositions thereof.

As used in this specification and unless otherwise indicated the substituent terms “alkyl”, “alkenyl”, “alkynyl”, “alkoxy”, “alkenyloxy”, and “alkynyloxy” used alone or as part of a larger moiety, includes straight or branched chains of at least one or two carbon atoms, as appropriate to the substituent, and preferably up to 12 carbon atoms, more preferably up to ten carbon atoms, most preferably up to seven carbon atoms, wherein “alkenyl” has at least one carbon to carbon double bond, and “alkynyl” has at least one carbon to carbon triple bond. The term “aryl” refers to an aromatic ring structure, including fused rings, having six to ten carbon atoms. The term “heteroaryl” refers to an aromatic ring structure, including fused rings, having four to ten carbon atoms, and in which one or more of the atoms in the ring is other than carbon, for example, sulfur, oxygen, or nitrogen. The term “THF” refers to tetrahydrofuran. The term “DMSO” refers to methyl sulfoxide. The term “DMF” refers to N,N-dimethylformamide. The term p-TSA refers to para-toluenesulfonic acid monohydrate. The term “halogen” or “halo” refers to fluorine, bromine, iodine, or chlorine. The term “ambient temperature” or “room temperature” often abbreviated as “RT”, for example, in reference to a chemical reaction mixture temperature, refers to a temperature in the range of 20° C. to 30° C.

The compounds of formula I of the present invention can be synthesized by methods that are individually known to one skilled in the art from intermediate compounds readily available in commerce. Scheme 1 below illustrates a general procedure for synthesizing those compounds of formula I where, for example, R is OR¹⁶ and R¹⁶ is hydrogen; R¹, R², R⁴, R⁵, R⁶, R⁷ and R⁹ through R¹⁵, inclusively, are hydrogen; R³ and R⁵ are each OCF₃; A and B are each —O(CR¹⁸R¹⁹)_{q}— where R¹⁸ and R¹⁹ are each hydrogen and q in A and B is 1; and n and s are 0:

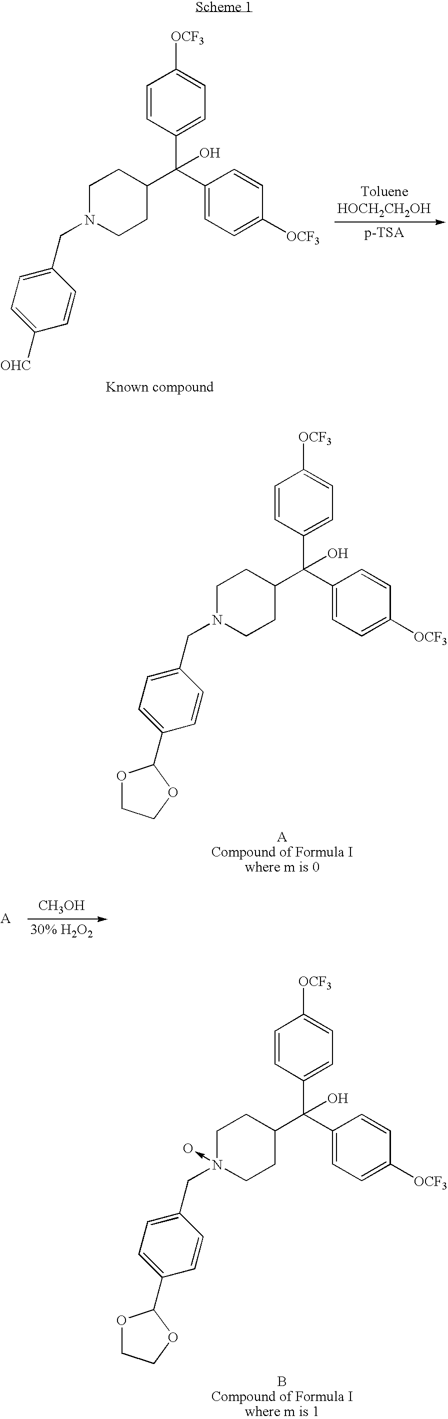

As depicted in Scheme 1 an appropriately substituted benzaldehyde, for example 4-({4-[bis(4-trifluoromethoxyphenyl)hydroxymethyl]piperidinyl}methyl)benzaldealdehyde, a known compound, was reacted under catalytic conditions with an appropriate optionally substituted alkylene glycol, such as ethylene glycol, to afford the corresponding ketal derivative Compound A, a compound of Formula I, for example, bis[4-(trifluoromethoxy)phenyl]{1-[(4-(1,3-dioxolan-2-yl)phenyl)methyl](4-piperidyl)}methan-1-ol. The so-prepared Compound A of formula I was optionally oxidized with, for example 30% hydrogen peroxide in an appropriate solvent, yielding the corresponding N-oxide Compound B, also a compound of formula I. Examples 1 and 2 set forth below provide detailed methods as to how compounds of formula A and B of the present invention, as depicted above, were prepared.

Compounds of formula I in which s is 1 and W is —OCR³⁰R³¹—, where R³⁰ and R³¹ are hydrogen, were prepared in a manner described in Scheme 2.

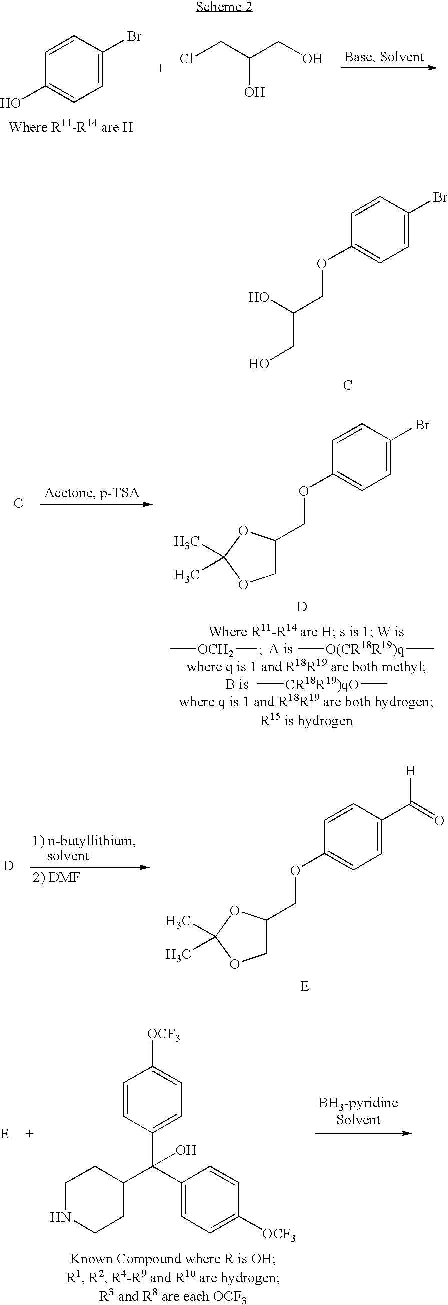

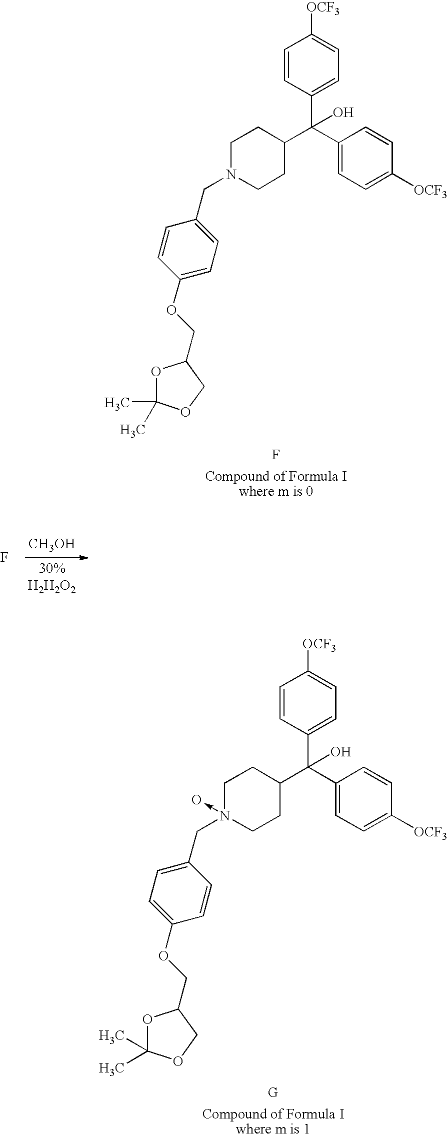

As depicted in Scheme 2 an appropriately substituted phenol, for example 4-bromophenol, a known compound, was reacted with an alkyl diol, for example, 3-chloro-1,2-propanediol, under basic conditions, to afford the phenoxy substituted diol intermediate C, for example, 3-(4-bromophenoxy)propane-1,2-diol. Intermediate C was reacted under catalytic conditions with an appropriate ketone, such as acetone, to produce the dioxolanyl intermediate D, for example, 1-[(2,2-dimethyl(1,3-dioxolan-4-yl))methoxy]-4-bromobenzene. Treatment of intermediate D first with n-butyllithium followed by DMF in an appropriate solvent afforded the benzaldehyde intermediate E, for example, 4-[(2,2-dimethyl-1,3-dioxolan-4-yl)methoxy]benzaldehyde. The reaction of intermediate E with borane-pyridine complex and an appropriately substituted piperidylmethanol, for example, bis[4-(trifluoromethoxy)phenyl]-4-piperidylmethan-1-ol provided the substituted piperidyl methanol, Compound F, for example, bis[4-(trifluoromethoxy)phenyl][1-({4-[2,2-dimethyl(1,3-dioxolan-4-yl)methoxy]phenyl}methyl)(4-piperidyl)]methan-1-ol, a compound of formula I. Compound F was oxidized with, for example, 30% hydrogen peroxide in an appropriate solvent yielding the corresponding N-oxide Compound G, also a compound of formula I. Example 3 set forth below provides a detailed method for the preparation of compounds of formula I; structures F and G.

Compounds of formula I in which A and B are each —O(CR¹⁸R¹⁹)_{q}— where one of R¹⁸ and R¹⁹ in either A or B is other then hydrogen and n and s are 0, were prepared in a manner described in Scheme 3.

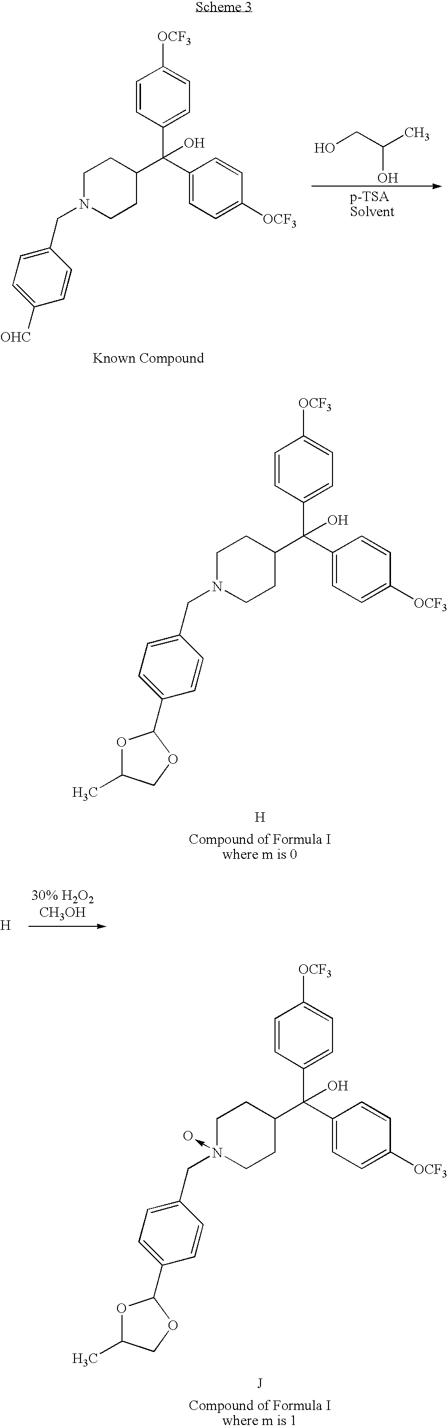

As depicted in Scheme 3 an appropriate formaldehyde, for example, 4-({4-[bis(4-(trifluoromethoxy)phenyl)hydroxymethyl]piperidinyl}methyl)benzaldehyde, a known compound, was reacted under catalytic conditions with an appropriate optionally substituted alkyldiol, such as 1,2-propanediol, to afford the corresponding ketal, Compound H, a compound of formula I, for example, bis[4-(trifluoromethoxy)phenyl](1-{[4-(4-methyl(1,3-dioxolan-2-yl))phenyl]methyl}(4-piperidyl))methan-1-ol. The so-prepared Compound H of Formula I was optionally oxidized with, for example 30% hydrogen peroxide in an appropriate solvent, to yield the corresponding N-oxide Compound J, also a compound of formula I. Example 4 set forth below provides detailed methods as to how compounds of formula I of the present invention, as depicted above in structures H and J, were prepared.

Compounds of formula I in which A and B are each —O(CR¹⁸R¹⁹)_{q}— where R¹⁸ and R¹⁹ are hydrogen; q in A and B are 1; R¹¹, R¹² and R¹³ are hydrogen; R¹⁵ is alkyl and n and s are 0, were prepared in a manner described in Scheme 4.

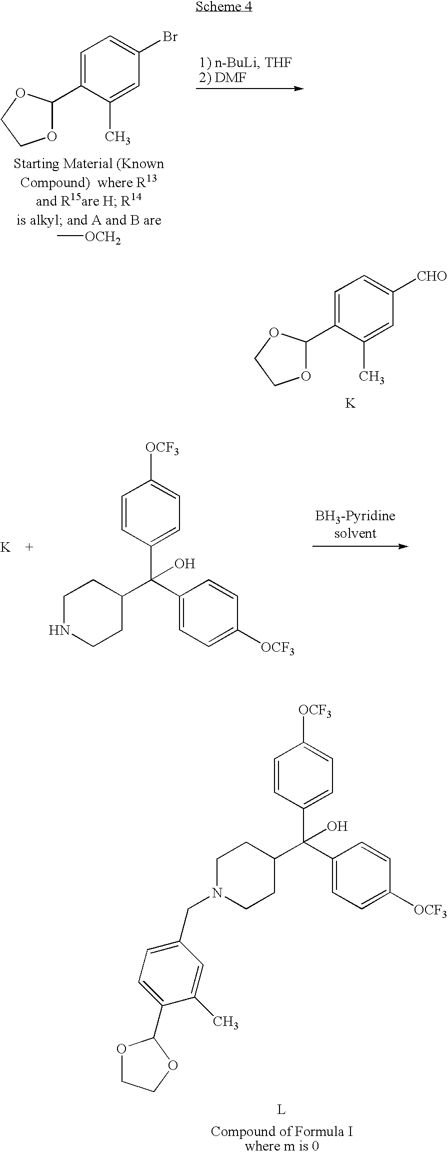

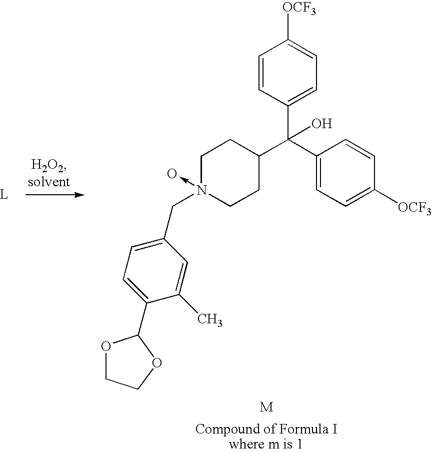

As depicted in Scheme 4 an appropriately substituted phenyl dioxolane, for example, 2-(4-bromo-2-methylphenyl)-1,3-dioxolane (known compound), was reacted first with n-butyllithium followed by DMF in an appropriate solvent to afford the benzaldehyde intermediate K, for example, 4-(1,3-dioxolan-2-yl)-3-methylbenzaldehyde. The reaction of intermediate K with borane-pyridine complex and an appropriately substituted piperidylmethanol, for example, bis[4-(trifluoromethoxy)phenyl]-4-piperidylmethan-1-ol, provided the substituted piperidyl methanol Compound L, for example, bis[4-(trifluoromethoxy)phenyl]{1-[(4-(1,3-dioxolan-2-yl)-3-methylphenyl)methyl](4-piperidyl)}methan-1-ol, a compound of Formula I. Compound L was oxidized with, for example, 30% hydrogen peroxide in an appropriate solvent to yield the corresponding N-oxide, Compound M, also a compound of formula I. Example 6 set forth below provides a detailed description of the preparation of compounds of formula I; structures L and M.

Scheme 5 below illustrates an alternative process for the preparation of compounds of formula I in which s is 1 and W is —OCR³⁰R³¹—, where R³⁰ and R³¹ are hydrogen:

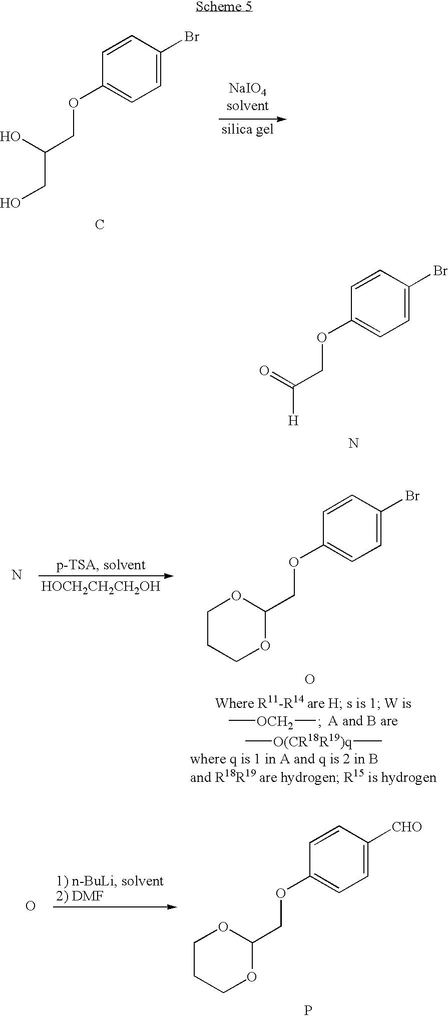

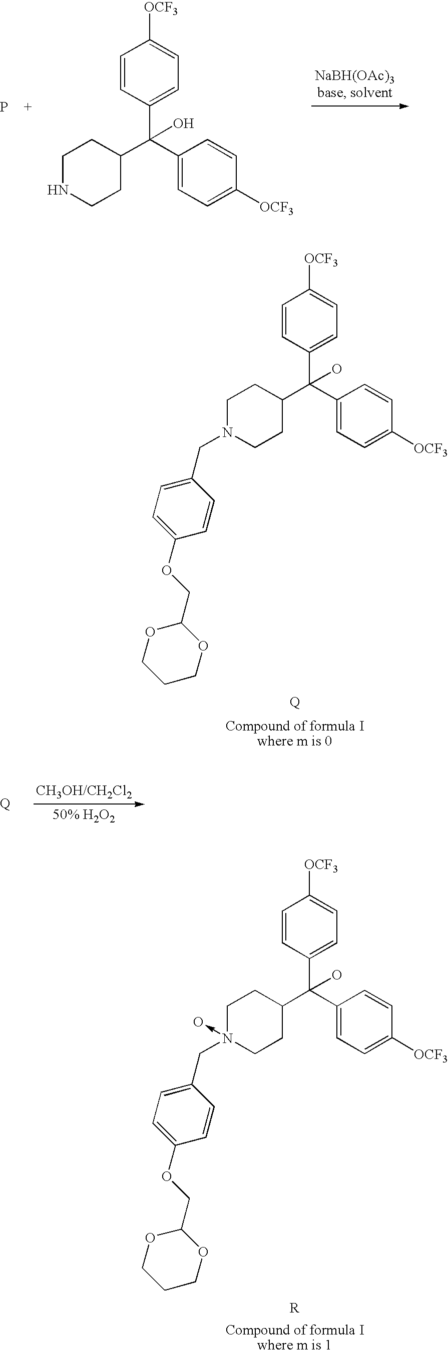

As depicted in Scheme 5, an appropriately substituted phenoxy diol, intermediate C, for example, 3-(4-bromophenoxy)propane-1,2-diol, was reacted with sodium periodate in the presence of silica gel in a solvent to produce the phenoxy ethan-1-one intermediate N, for example, 2-(4-bromophenoxy)ethan-1-one. The reaction of intermediate N under catalytic conditions with an appropriate optionally substituted alkylene glycol, such as 1,3-propanediol, afforded the corresponding dioxanylmethoxy benzene intermediate 0, for example, 1-bromo 4-(1,3-dioxan-2-ylmethoxy)benzene. Treatment of intermediate 0 first with n-butlylithium followed by DMF in an appropriate solvent afforded the benzaldehyde intermediate P, for example, 4-(1,3-dioxan-2-ylmethoxy)benzaldehyde. The reaction of intermediate P under basic conditions with sodium triacetoxyborohydride and an appropriately substituted piperidylmethanol, for example, bis[4-(trifluoromethoxy)phenyl]-4-piperidylmethan-1-ol provided the substituted piperidyl methanol Compound Q, for example, bis[4-(trifluoromethoxy)phenyl]{1-[(4-(1,3-dioxan-2-yl)methoxy)phenyl)methyl] (4-piperidyl)}methan-1-ol. The so-prepared Compound Q of formula I was optionally oxidized with, for example, 50% hydrogen peroxide in an appropriate solvent, to yield the corresponding N-oxide, Compound R, also a compound of formula I. Example 5 set forth below provides a detailed description for the preparation of compounds of formula I; structures Q and R.

One skilled in the art will, of course, recognize that the formulation and mode of application of a toxicant may affect the activity of the material in a given application. Thus, for agricultural use the present insecticidal compounds may be formulated as a granular of relatively large particle size (for example, 8/16 or 4/8 US Mesh), as water-soluble or water-dispersible granules, as powdery dusts, as wettable powders, as emulsifiable concentrates, as aqueous emulsions, as solutions, or as any of other known types of agriculturally-useful formulations, depending on the desired mode of application. It is to be understood that the amounts specified in this specification are intended to be approximate only, as if the word “about” were placed in front of the amounts specified.

These insecticidal compositions may be applied either as water-diluted sprays, or dusts, or granules to the areas in which suppression of insects is desired. These formulations may contain as little as 0.1%, 0.2% or 0.5% to as much as 95% or more by weight of active ingredient.

Dusts are free flowing admixtures of the active ingredient with finely divided solids such as talc, natural clays, kieselguhr, flours such as walnut shell and cottonseed flours, and other organic and inorganic solids which act as dispersants and carriers for the toxicant; these finely divided solids have an average particle size of less than about 50 microns. A typical dust formulation useful herein is one containing 1.0 part or less of the insecticidal compound and 99.0 parts of talc.

Wettable powders, also useful formulations for insecticides, are in the form of finely divided particles that disperse readily in water or other dispersant. The wettable powder is ultimately applied to the locus where insect control is needed either as a dry dust or as an emulsion in water or other liquid. Typical carriers for wettable powders include Fuller's earth, kaolin clays, silicas, and other highly absorbent, readily wet inorganic diluents. Wettable powders normally are prepared to contain about 5-80% of active ingredient, depending on the absorbency of the carrier, and usually also contain a small amount of a wetting, dispersing or emulsifying agent to facilitate dispersion. For example, a useful wettable powder formulation contains 80.0 parts of the insecticidal compound, 17.9 parts of Palmetto clay, and 1.0 part of sodium lignosulfonate and 0.3 part of sulfonated aliphatic polyester as wetting agents. Additional wetting agent and/or oil will frequently be added to a tank mix for to facilitate dispersion on the foliage of the plant.

Other useful formulations for insecticidal applications are emulsifiable concentrates (ECs) which are homogeneous liquid compositions dispersible in water or other dispersant, and may consist entirely of the insecticidal compound and a liquid or solid emulsifying agent, or may also contain a liquid carrier, such as xylene, heavy aromatic naphthas, isphorone, or other non-volatile organic solvents. For insecticidal application these concentrates are dispersed in water or other liquid carrier and normally applied as a spray to the area to be treated. The percentage by weight of the essential active ingredient may vary according to the manner in which the composition is to be applied, but in general comprises 0.5 to 95% of active ingredient by weight of the insecticidal composition.

Flowable formulations are similar to ECs, except that the active ingredient is suspended in a liquid carrier, generally water. Flowables, like ECs, may include a small amount of a surfactant, and will typically contain active ingredients in the range of 0.5 to 95%, frequently from 10 to 50%, by weight of the composition. For application, flowables may be diluted in water or other liquid vehicle, and are normally applied as a spray to the area to be treated.

Typical wetting, dispersing or emulsifying agents used in agricultural formulations include, but are not limited to, the alkyl and alkylaryl sulfonates and sulfates and their sodium salts; alkylaryl polyether alcohols; sulfated higher alcohols; polyethylene oxides; sulfonated animal and vegetable oils; sulfonated petroleum oils; fatty acid esters of polyhydric alcohols and the ethylene oxide addition products of such esters; and the addition product of long-chain mercaptans and ethylene oxide. Many other types of useful surface-active agents are available in commerce. Surface-active agents, when used, normally comprise 1 to 15% by weight of the composition.

Other useful formulations include suspensions of the active ingredient in a relatively non-volatile solvent such as water, corn oil, kerosene, propylene glycol, or other suitable solvents.

Still other useful formulations for insecticidal applications include simple solutions of the active ingredient in a solvent in which it is completely soluble at the desired concentration, such as acetone, alkylated naphthalenes, xylene, or other organic solvents. Granular formulations, wherein the toxicant is carried on relative coarse particles, are of particular utility for aerial distribution or for penetration of cover crop canopy. Pressurized sprays, typically aerosols wherein the active ingredient is dispersed in finely divided form as a result of vaporization of a low-boiling dispersant solvent carrier may also be used. Water-soluble or water-dispersible granules are free flowing, non-dusty, and readily water-soluble or water-miscible. In use by the farmer on the field, the granular formulations, emulsifiable concentrates, flowable concentrates, aqueous emulsions, solutions, etc., may be diluted with water to give a concentration of active ingredient in the range of say 0.1% or 0.2% to 1.5% or 2%.

The active insecticidal compounds of this invention may be formulated and/or applied with one or more additional compounds. Such combinations may provide certain advantages, such as, without limitation, exhibiting synergistic effects for greater control of insect pests, reducing rates of application of insecticide thereby minimizing any impact to the environment and to worker safety, controlling a broader spectrum of insect pests, safening of crop plants to phytotoxicity, and improving tolerance by non-pest species, such as mammals and fish.

Additional compounds include, without limitation, other pesticides, plant growth regulators, fertilizers, soil conditioners, or other agricultural chemicals. In applying an active compound of this invention, whether formulated alone or with other agricultural chemicals, an effective amount and concentration of the active compound is of course employed; the amount may vary in the range of, e.g. about 0.001 to about 3 kg/ha, preferably about 0.03 to about 1 kg/ha. For field use, where there are losses of insecticide, higher application rates (e.g., four times the rates mentioned above) may be employed.

When the active insecticidal compounds of the present invention are used in combination with one or more additional compounds, e.g., with other pesticides such as herbicides, the herbicides include, without limitation, for example: N-(phosphonomethyl)glycine (“glyphosate”); aryloxyalkanoic acids such as (2,4-dichlorophenoxy)acetic acid (“2,4-D”), (4-chloro-2-methylphenoxy)acetic acid (“MCPA”), (+/−)-2-(4-chloro-2-methylphenoxy)propanoic acid (“MCPP”); ureas such as N,N-dimethyl-N′-[4-(1-methylethyl)phenyl]urea (“isoproturon”); imidazolinones such as 2-[4,5-dihydro-4-methyl-4-(1-methylethyl)-5-oxo-1H-imidazol-2-yl]-3-pyridinecarboxylic acid (“imazapyr”), a reaction product comprising (+/−)-2-[4,5-dihydro-4-methyl-4-(1-methylethyl)-5-oxo-1H-imidazol-2-yl]-4-methylbenzoic acid and (+/−)₂-[4,5-dihydro-4-methyl-4-(1-methylethyl)-5-oxo-1H-imidazol-2-yl]-5-methylbenzoic acid (“imazamethabenz”), (+/−)-2-[4,5-dihydro-4-methyl-4-(1-methylethyl)-5-oxo-1H-imidazol-2-yl]-5-ethyl-3-pyridinecarboxylic acid (“imazethapyr”), and (+/−)-2-[4,5-dihydro-4-methyl-4-(1-methylethyl)-5-oxo-1H-imidazol-2-yl]-3-quinolinecarboxylic acid (“imazaquin”); diphenyl ethers such as 5-[2-chloro-4-(trifluoromethyl)phenoxy]-2-nitrobenzoic acid (“acifluorfen”), methyl 5-(2,4-dichlorophenoxy)-2-nitrobenzoate (“bifenox”), and 5-[2-chloro-4-(trifluoromethyl)phenoxy]-N-(methylsulfonyl)-2-nitrobenzamide (“fomasafen”); hydroxybenzonitriles such as 4-hydroxy-3,5-diiodobenzonitrile (“ioxynil”) and 3,5-dibromo-4-hydroxybenzonitrile (“bromoxynil”); sulfonylureas such as 2-[[[[(4-chloro-6-methoxy-2-pyrimidinyl)amino]carbonyl]amino]sulfonyl]benzoic acid (“chlorimuron”), 2-chloro-N—[[(4-methoxy-6-methyl-1,3,5-triazin-2-yl)amino]carbonyl]benzenesulfonamide (achlorsulfuron”), 2-[[[[[(4,6-dimethoxy-2-pyrimidinyl)amino]carbonyl]amino]sulfonyl]methyl]benzoic acid (“bensulfuron”), 2[[[[(4,6-dimethoxy-2-pyrimidinyl)amino]carbonyl]amino]sulfonyl]-1-methyl-1H-pyrazol-4-carboxylic acid (“pyrazosulfuron”), 3-[[[[(4-methoxy-6-methyl-1,3,5-triazin-2-yl)amino]carbonyl]amino]sulfonyl]-2-thiophenecarboxylic acid (“thifensulfuron”), and 2-(2-chloroethoxy)-N[[(4-methoxy-6-methyl-1,3,5-triazin-2-yl)amino]carbonyl]benzenesulfonamide (“triasulfuron”); 2-(4-aryloxy-phenoxy)alkanoic acids such as (+/−)-2-[4-[(6-chloro-2-benzoxazolyl)oxy]phenoxy]-propanoic acid (fenoxaprop”), (+/−)-2-[4[[5-(trifluoromethyl)-2-pyridinyl]oxy]-phenoxy]propanoic acid (“fluazifop”), (+/−)-2-[4-(6-chloro-2-quinoxalinyl)oxy]-phenoxy]propanoic acid (“quizalofop”), and (+/−)-2-[(2,4-dichlorophenoxy)phenoxy]propanoic acid (“diclofop”); benzothiadiazinones such as 3-(1-methylethyl)-1H-1,2,3-benzothiadiazin-4(3H)-one-2,2-dioxide (“bentazone”); 2-chloroacetanilides such as N-(butoxymethyl)-2-chloro-N-(2,6-diethylphenyl)acetamide (“butachlor”), 2-chloro-N-(2-ethyl-6-methylphenyl)-N-(2-methoxy-1-methylethyl)acetamide (“metolachlor”), 2-chloro-N-(ethoxymethyl)-N-(2-ethyl-6-methylphenyl)acetamide (“acetochlor”), and (RS)-2-chloro-N-(2,4-dimethyl-3-thienyl)-N-(2-methoxy-1-methylethyl)acetamide (“dimethenamide”); arenecarboxylic acids such as 3,6-dichloro-2-methoxybenzoic acid (“dicamba”); pyridyloxyacetic acids such as [(4-amino-3,5-dichloro-6-fluoro-2-pyridinyl)oxy]acetic acid (“fluoroxypyr”); aryl triazolinones such as 1H-1,2,4-triazol-1-carboxamide (“amicarbazone”), 1,2,4-triazolo[4,3-a]pyridine-3(2H)-one (“azafenidin”), N-(2,4-dichloro-5-[4-(difluoromentyl)-4,5-dihydro-3-methyl-5-oxo-1H-1,2,4-triazol-1-yl]phenyl)methanesulfonamide (“sulfentrazone”) and ethyl α, 2-dichloro-5-[4-(difluoromethyl)-4,5-dihydro-3-methyl-5-oxo-1H-1,2,4-triazol-1-yl]-4-fluorobenzenepropanoate (“carfentrazone-ethyl”); isoxazolidinones such as 2-[(2-chlorophenyl)methyl]-4,4-dimethyl-3-isoxazoline (“clomazone”); and other herbicides.

When the active insecticidal compounds of the present invention are used in combination with one or more additional compounds, e.g., with other pesticides such as other insecticides, the other insecticides include, for example: organophosphate insecticides, such as chlorpyrifos, diazinon, dimethoate, malathion, parathion-methyl, and terbufos; pyrethroid insecticides, such as fenvalerate, deltamethrin, fenpropathrin, cyfluthrin, flucythrinate, alpha-cypermethrin, beta-cypermethrin, zeta-cypermethrin, bifenthrin, resolved cyhalothrin, etofenprox, esfenvalerate, tralomehtrin, tefluthrin, cycloprothrin, betacyfluthrin, and acrinathrin; carbamate insecticides, such as aldecarb, carbaryl, carbofuran, and methomyl; organochlorine insecticides, such as endosulfan, endrin, heptachlor, and lindane; benzoylurea insecticides, such as diflubenuron, triflumuron, teflubenzuron, chlorfluazuron, flucycloxuron, hexaflumuron, flufenoxuron, and lufenuron; and other insecticides, such as amitraz, clofentezine, fenpyroximate, hexythiazox, spinosad, imidacloprid, flonicamid and pyridalyl.

When the active insecticidal compounds of the present invention are used in combination with one or more additional compounds, e.g., with other pesticides such as fungicides, the fungicides include, for example: benzimidazole fungicides, such as benomyl, carbendazim, thiabendazole, and thiophanate-methyl; 1,2,4-triazole fungicides, such as epoxyconazole, cyproconazole, flusilazole, flutriafol, propiconazole, tebuconazole, triadimefon, and triadimenol; substituted anilide fungicides, such as metalaxyl, oxadixyl, procymidone, and vinclozolin; organophosphorus fungicides, such as fosetyl, iprobenfos, pyrazophos, edifenphos, and tolclofos-methyl; morpholine fungicides, such as fenpropimorph, tridemorph, and dodemorph; other systemic fungicides, such as fenarimol, imazalil, prochloraz, tricyclazole, and triforine; dithiocarbamate fungicides, such as mancozeb, maneb, propineb, zineb, and ziram; non-systemic fungicides, such as chlorothalonil, dichlofluanid, dithianon, and iprodione, captan, dinocap, dodine, fluazinam, gluazatine, PCNB, pencycuron, quintozene, tricylamide, and validamycin; inorganic fungicides, such as copper and sulphur products, and other fungicides.

When the active insecticidal compounds of the present invention are used in combination with one or more additional compounds, e.g., with other pesticides such as nematicides, the nematicides include, for example: carbofuran, carbosulfan, turbufos, aldecarb, ethoprop, fenamphos, oxamyl, isazofos, cadusafos, and other nematicides.

When the active insecticidal compounds of the present invention are used in combination with one or more additional compounds, e.g., with other materials such as plant growth regulators, the plant growth regulators include, for example: maleic hydrazide, chlormequat, ethephon, gibberellin, mepiquat, thidiazon, inabenfide, triaphenthenol, paclobutrazol, unaconazol, DCPA, prohexadione, trinexapac-ethyl, and other plant growth regulators.

Soil conditioners are materials which, when added to the soil, promote a variety of benefits for the efficacious growth of plants. Soil conditioners are used to reduce soil compaction, promote and increase effectiveness of drainage, improve soil permeability, promote optimum plant nutrient content in the soil, and promote better pesticide and fertilizer incorporation. When the active insecticidal compounds of the present invention are used in combination with one or more of second compounds, e.g., with other materials such as soil conditioners, the soil conditioners include organic matter, such as humus, which promotes retention of cation plant nutrients in the soil; mixtures of cation nutrients, such as calcium, magnesium, potash, sodium, and hydrogen complexes; or microorganism compositions which promote conditions in the soil favorable to plant growth. Such microorganism compositions include, for example, bacillus, pseudomonas, azotobacter, azospirillum, rhizobium, and soil-borne cyanobacteria.

Fertilizers are plant food supplements, which commonly contain nitrogen, phosphorus, and potassium. When the active insecticidal compounds of the present invention are used in combination with one or more of second compounds, e.g., with other materials such as fertilizers, the fertilizers include nitrogen fertilizers, such as ammonium sulfate, ammonium nitrate, and bone meal; phosphate fertilizers, such as superphosphate, triple superphosphate, ammonium sulfate, and diammonium sulfate; and potassium fertilizers, such as muriate of potash, potassium sulfate, and potassium nitrate, and other fertilizers.

The following examples further illustrate the present invention, but, of course, should not be construed as in any way limiting its scope. The examples are organized to present protocols for the synthesis of the compounds of formula I of the present invention, set forth a list of such synthesized species, and set forth certain biological data indicating the efficacy of such compounds.

### EXAMPLE 1

### This example illustrates one protocol for the preparation of bis[4-(trifluoromethoxy)-phenyl]{1-[(4-(1,3-dioxolan-2-yl)phenyl)methyl] (4-piperidyl)methan-1-ol (Compound 1)

A stirred solution of 0.5 gram (0.0009 mole) of 4-({4-[bis(4-(trifluoromethoxy)phenyl)hydroxymethyl]piperidinyl}methyl)benzaldehyde (known compound; commercially available), 6 mL (excess) of ethylene glycol and a catalytic amount of p-TSA in 100 mL of toluene was heated at reflux for six hours while the water by-product was collected in a Dean-Stark trap. After this time the reaction mixture was cooled and poured into an aqueous solution of sodium bicarbonate. The organic layer was separated and washed with an aqueous solution saturated with sodium chloride. The organic layer was then dried with sodium sulfate, filtered, and the filtrate was concentrated under reduced pressure to a residue. The residue was purified with column chromatography on silica gel. Elution was accomplished using mixtures of ethyl acetate in hexane as eluants. The appropriate fractions were combined and concentrated under reduced pressure, yielding 0.5 gram of Compound 1. The NMR spectrum was consistent with the proposed structure.

### EXAMPLE 2

### This example illustrates one protocol for the preparation of bis[4-(trifluoromethoxy)-phenyl]{1-[(4-(1,3-dioxolan-2-yl)phenyl)methyl](4-piperidyl)methan-1-ol, N-oxide (Compound 2)

A solution of 0.14 gram (0.0002 mole) of bis[4-(trifluoromethoxy)phenyl]{1-[(4-(1,3-dioxolan-2-yl)phenyl)methyl](4-piperidyl)methan-1-ol (Compound 1) and 1.8 mL (excess) of aqueous 30% hydrogen peroxide in 7 mL of methanol was stirred at ambient temperature during a 16 hour period. After this time the methanol was removed under reduced pressure, and the concentrate was extracted with methylene chloride. The extract was dried with sodium sulfate, filtered, and the filtrate was concentrated under reduced pressure, yielding 0.11 gram of Compound 2, mp 98-108° C. The NMR spectrum was consistent with the proposed structure.

### EXAMPLE 3

### This example illustrates one protocol for the preparation of bis[4-(trifluoromethoxy)-phenyl][1-({4-[(2,2-dimethyl(1,3-dioxolan-4-yl))methoxy]phenyl}methyl)(4-piperidyl)]methan-1-ol (Compound 189) and the corresponding N-oxide (Compound 190)

Step A Preparation of 3-(4-bromophenoxy)propane-1,2-diol as an intermediate

To a stirred solution of 4.0 grams (0.023 mole) of 4-bromophenol in 70.0 mL of acetonitrile was added 16.0 grams (0.116 mole) of potassium carbonate and 2.56 grams (0.023 mole) of 3-chloro-1,2-propanediol. The reaction mixture was heated at reflux for 16 hours. The reaction mixture was cooled and the solvent removed by evaporation under reduced pressure leaving a solid residue. The solid residue was partitioned between ethyl acetate and water. The organic phase was washed with an aqueous 10% sodium hydroxide solution, dried with sodium sulfate and filtered. The filtrate was concentrated under reduced pressure to yield 5.15 grams of 3-(4-bromophenoxy)propane-1,2-diol as a solid, melting point 69-75° C. The NMR spectrum was consistent with the proposed structure.
Step B Preparation of 1-[(2,2-dimethyl(1,3-dioxolan-4-yl))methoxy]-4-bromobenzene as an intermediate

A mixture of 1.0 gram (0.004 mole) of 3-(4-bromophenoxy)propane-1,2-diol, 0.38 gram (0.002 mole) of p-TSA and 0.7 gram of 4 Angstrom molecular sieves (8-12 mesh) in 10.6 mL of acetone was stirred and heated at reflux for 20 hours. The reaction mixture was cooled and the solvent was removed by evaporation under reduced pressure leaving a residue. The residue was extracted with ethyl acetate. The organic extract was washed with an aqueous saturated sodium carbonate solution, dried with sodium sulfate and filtered. The filtrate was concentrated under reduced pressure to yield 0.99 gram of 1-[(2,2-dimethyl(1,3-dioxolan-4-yl))methoxy]-4-bromobenzene. The NMR spectrum was consistent with the proposed structure.
Step C Preparation of 4-[(2,2-dimethyl-1,3-dioxolan-4-yl)methoxy]benzaldehyde as an intermediate

To a stirred, cold (minus 78° C.), solution of 0.99 gram (0.0034 mole) of 1-[(2,2-dimethyl(1,3-dioxolan-4-yl))methoxy]-4-bromobenzene in 6.9 mL of THF was added 1.52 mL of a 2.5 molar solution of n-butyllithium in hexanes (0.0038 mole). The reaction mixture was stirred for 8 minutes and 0.4 mL (0.0052 mole) of DMF was added. The reaction mixture stirred at minus 78° C. for 10 minutes at which time approximately 7 mL of water was added. The reaction mixture was allowed to warm to ambient temperature and was extracted with ethyl acetate. The extract was dried with sodium sulfate, filtered and the filtrate concentrated under reduced pressure to yield 0.9 gram of an oil. NMR analysis of the oil indicated that it consisted of 60% of 4-[(2,2-dimethyl-1,3-dioxolan-4-yl)methoxy]benzaldehyde.
Step D Preparation of bis[4-(trifluoromethoxy)phenyl][1-({4-[(2,2-dimethyl-(1,3-dioxolan-4-yl))methoxy]phenyl}methyl)(4-piperidyl)]methan-1-ol (Compound 189)

A mixture of 0.81 gram of the oil prepared in Step C (0.002 mole of 4-[(2,2-dimethyl-1,3-dioxolan-4-yl)methoxy]benzaldehyde (known compound), 0.9 gram (0.002 mole) of bis(4-trifluoromentoxyphenyl)-4-piperidylmethan-1-ol and 0.31 mL (0.0028 mole) of borane-pyridine complex in 3.95 mL of ethanol was stirred in a sealed container for 16 hours. The reaction mixture was diluted with 7 mL of an aqueous saturated sodium chloride solution and 7 mL of water. The mixture was extracted with ethyl acetate, the extract concentrated under reduced pressure to a residue. The residue was purified by column chromatography on silica gel, eluting with mixtures of hexanes, methylene chloride and acetone. The appropriate fractions were combined and concentrated under reduced pressure to yield 0.36 gram of Compound 189. The NMR spectrum was consistent with the proposed structure.
Step E Preparation of bis[4-(trifluoromethoxy)phenyl][1-({4-[(2,2-dimethyl-(1,3-dioxolan-4-yl))methoxy]phenyl}methyl)(4-piperidyl)]methan-1-ol, N-oxide (Compound 190)

A mixture of 0.28 gram (0.00043 mole) of bis[4-(trifluoromethoxy)phenyl][1-({4-[(2,2-dimethyl-(1,3-dioxolan-4-yl))methoxy]phenyl}methyl)(4-piperidyl)]methan-1-ol (Compound 189) and 3.3 mL of aqueous 30% hydrogen peroxide in 13 mL of methanol was stirred at 35° C. for 16 hours. The mixture was concentrated under reduced pressure to a milky liquid residue. The residue was added to a mixture of sodium sulfate and methylene chloride. The liquid portion of the mixture was filtered through a pad of sodium sulfate and the filtrate was concentrated to a white semi-solid residue. The residue was purified by column chromatography on silica gel, eluting with mixtures of methanol and methylene chloride. The appropriate fractions were combined and concentrated under reduced pressure to yield 0.17 gram of Compound 190 as a solid, melting point 96-107° C. The NMR spectrum was consistent with the proposed structure.

### EXAMPLE 4

### This example illustrates one protocol for the preparation of bis[4-(trifluoromethoxy)-phenyl](1-{[(4-(4-methyl(1,3-dioxolan-2-yl))phenyl]methyl}(4-piperidyl)) methan-1-ol (Compound 3) and the corresponding N-oxide (Compound 9)

Step A Preparation of bis[4-(trifluoromethoxy)phenyl] (1-{[4-(4-methyl(1,3-dioxolan-2-yl)phenyl]methyl}(4-piperidyl))methan-1-ol (Compound 3)

A stirred mixture of 0.3 gram (0.00054 mole) of 4-({4-[bis(4-(trifluoromethoxy)phenyl)hydroxymethyl]piperidinyl}methyl)benzaldehyde (known compound), 0.4 mL of 1,2-propanediol and a catalytic amount of p-TSA in 27 mL of toluene was heated at reflux for 16 hours while the water by-product was collected in a Dean-Stark trap. The reaction mixture was cooled then concentrated under reduced pressure to a liquid residue. The residue was dissolved in 20 mL of anhydrous methylene chloride to which 0.078 gram (0.0019 mole) of sulfonylhydrazide resin was added. The mixture stirred at ambient temperature for about 18 hours at which time the mixture was filtered. The filtrate was washed in succession with 30 mL of an aqueous saturated sodium carbonate solution and 30 mL of an aqueous saturated sodium chloride solution, dried with sodium sulfate, and was filtered. The filtrate was concentrated under reduced pressure to a yellowish residue. The residue was purified by column chromatography on silica gel, eluting with mixtures of acetone and methylene chloride. The appropriate fractions were combined and concentrated under reduced pressure to yield 0.22 gram of Compound 3. The NMR spectrum was consistent with the proposed structure.
Step B Preparation of bis[4-(trifluoromethoxy)phenyl](1-{[(4-(4-methyl(1,3-dioxolan-2-yl))phenyl]methyl}(4-piperidyl)) methan-1-ol, N-oxide (Compound 9)

In a manner similar to Example 2, the reaction of 0.22 gram (0.0037 mole) of bis[4-(trifluoromethoxy)phenyl](1-{[(4-(4-methyl(1,3-dioxolan-2-yl))phenyl]methyl}(4-piperidyl)) methan-1-ol with 2.8 mL of aqueous 30% hydrogen peroxide in 12 mL of methanol produced 0.21 gram of Compound 9 as a solid. The NMR spectrum was consistent with the proposed structure.

### EXAMPLE 5

### This example illustrates one protocol for the preparation of bis[4-(trifluoromethoxy)-phenyl](1-{[4-(1,3-dioxan-2-ylmethoxy)phenyl]methyl}(4-piperidyl))methan-1-ol (Compound 199) and the corresponding N-oxide (Compound 200)

Step A Preparation of 2-(4-bromophenoxy)ethan-1-one as an intermediate

A solution of 11.77 grams (0.055 mole) of sodium periodate in 20 mL of water was added dropwise to a stirred mixture of 85 grams (200-300 mesh, 60 angstrom) of silica gel in 340 mL of methylene chloride. To this mixture was added dropwise, a solution of 10.5 grams (0.042 mole) of 3-(4-bromophenoxy)propane-1,2-diol dissolved in 85 mL of methylene chloride. Upon complete addition the reaction mixture was stirred at ambient temperature for one hour. The reaction mixture was filtered and the filter cake rinsed with methanol. The filtrate and the rinse were combined and then concentrated under reduced pressure to leave a residue. The residue was partitioned between methylene chloride and water and the aqueous phase was extracted with methylene chloride. The organic phases were combined, dried with magnesium sulfate and filtered. The filtrate was condensed under reduced pressure to a residue. This residue was purified by column chromatography on silica gel, eluting with mixtures of hexanes and ethyl acetate. The appropriate fractions were combined and concentrated under reduced pressure to yield 9.1 grams of a liquid. NMR analysis of the liquid indicated that it was a mixture consisting of 40% 2-(4-bromophenoxy)ethan-1-one and 60% 2-(4-bromophenoxy)ethane-1,1-diol.
Step B Preparation of 1-bromo-4-(1,3-dioxan-2-yl)methoxybenzene as an intermediate

A mixture of 2.2 grams of the liquid prepared in Step A, 0.2 gram (0.001 mole) of p-TSA and 3.88 grams (0.051 mole) of 1,3-propanediol in 100 mL of toluene was stirred and heated at reflux for 20 hours while the water by-product was collected in a Dean-Stark trap. After this time the reaction mixture was cooled and diluted with ethyl acetate. The mixture was washed with water, dried with magnesium sulfate and filtered. The filtrate was condensed under reduced pressure to yield 2.8 gram of 1-bromo-4-(1,3-dioxan-2-yl)methoxybenzene. The NMR spectrum was consistent with the proposed structure.
Step C Preparation of 4-(1,3-dioxan-2-ylmethoxy)benzaldehyde as an intermediate

To a stirred, cold (minus 78° C.), solution of 1.74 gram (0.0064 mole) of 1-bromo-4-(1,3-dioxan-2-yl)methoxybenzene in 12.7 mL of THF was added 4.38 mL of a 1.6 molar solution of n-butyllithium in hexanes (0.007 mole). The reaction mixture was stirred for 30 minutes and 0.64 mL (0.007 mole) of DMF was added.

The reaction mixture stirred at minus 78° C. for one hour at which time the reaction mixture was poured into water. The mixture was extracted with two portions of ethyl acetate. The extracts were combined, dried with magnesium sulfate, filtered and the filtrate concentrated under reduced pressure to yield 1.3 grams of 4-(1,3-dioxan-2-ylmethoxy)benzaldehyde as an oil. The NMR spectrum was consistent with the proposed structure.
Step D Preparation of bis[4-(trifluoromethoxy)phenyl] (1-{[4-(1,3-dioxan-2-ylmethoxy)phenyl]methyl}(4-piperidyl))methan-1-ol (Compound 199)

A mixture of 0.25 gram (0.001 mole) of 4-(1,3-dioxan-2-ylmethoxy)benzaldehyde, 0.44 gram (0.001 mole) of bis[4-(trifluoromethoxy)phenyl}-4-piperidylmethan-1-ol, 0.43 gram (0.002 mole) of sodium triacetoxyborohydride and 0.5 mL (0.0035 mole) of triethylamine in 4.0 mL of THF was stirred at ambient temperature for 20 hours. The reaction mixture was diluted with water and then extracted with two 100 mL portions of ethyl acetate. The extracts were combined, dried with magnesium sulfate and filtered. The filtrate was concentrated under reduced pressure to a residue. The residue was purified by column chromatography on silica gel, eluting with mixtures of methylene chloride and acetone. The appropriate fractions were combined and concentrated under reduced pressure to yield 0.45 gram of Compound 199 as an oil. The NMR spectrum was consistent with the proposed structure.
Step E Preparation bis(4-trifluoromethoxyphenyl])(1-{[4-(1,3-dioxan-2-ylmethoxy)phenyl]methyl}(4-piperidyl))methan-1-ol, N-oxide (Compound 200)

A mixture of 0.3 gram (0.00046 mole) of bis[4-(trifluoromethoxy)phenyl](1-{[4-(1,3-dioxan-2-ylmethoxy)phenyl]methyl}(4-piperidyl))methan-1-ol (Compound 199) and 0.5 mL of aqueous 50% hydrogen peroxide in 4 mL of methanol/methylene chloride (50/50 mixture) was stirred at ambient temperature for 20 hours. The mixture was concentrated under reduced pressure to yield 0.3 gram of Compound 190 as a solid. The NMR spectrum was consistent with the proposed structure.

### EXAMPLE 6

### This example illustrates one protocol for the preparation of bis[4-(trifluoromethoxy)phenyl]{1-[(4-((1,3-dioxolan-2-yl)-3-methylphenyl)methyl](4-piperidyl)}methan-1-ol (Compound 169) and the corresponding N-oxide (Compound 170)

Step A Preparation of 4-(1,3-dioxolan-2-yl)-3-methylbenzaldehyde as an intermediate

To a stirred, cold (minus 78° C.), solution of 0.8 gram (0.0033 mole) of 2-(4-bromo-2-methylphenyl)-1,3-dioxolane (known compound) in 6.0 mL of THF, under a dry nitrogen atmosphere, was added 1.3 mL of a 2.5 molar solution of n-butyllithium in hexanes (0.0033 mole). The reaction mixture was stirred for 10 minutes and 0.39 mL (0.005 mole) of DMF was added. The reaction mixture stirred at minus 78° C. for 15 minutes at which time approximately 2 mL of water was added. The reaction mixture was allowed to warm to ambient temperature and was extracted with ethyl acetate. The extract was washed with two 30 mL portions of an aqueous saturated sodium chloride solution, dried with sodium sulfate, filtered and the filtrate concentrated under reduced pressure to an oil residue. The residue was purified by column chromatography on silica gel, eluting with mixtures of ethyl acetate and hexanes. The appropriate fractions were combined and concentrated under reduced pressure to yield 0.47 gram of 4-(1,3-dioxolan-2-yl)-3-methylbenzaldehyde as an oil. The NMR spectrum was consistent with the proposed structure.
Step B Preparation of bis[4-(trifluoromethoxy)phenyl]{1-[(4-((1,3-dioxolan-2-yl)-3-methylphenyl)methyl](4-piperidyl)}methan-1-ol (Compound 169)

In a manner similar to Example 3, Step D, the reaction of 0.47 gram (0.0024 mole of 4-(1,3-dioxolan-2-yl)-3-methylbenzaldehyde, 1.1 grams ((0.0024 mole) of bis[4-(trifluoromethoxy)phenyl)-4-piperidylmethan-1-ol, 0.33 mL (0.0024 mole) of triethylamine and 0.39 mL (0.0031 mole) of borane-pyridine complex in 8 mL of ethanol produced 0.92 gram of Compound 169 as a foam, melting point 57-60° C. The NMR spectrum was consistent with the proposed structure.
Step C Preparation of bis[4-(trifluoromethoxy)phenyl]{1-[(4-((1,3-dioxolan-2-yl)-3-methylphenyl)methyl](4-piperidyl)}methan-1-ol, N-oxide (Compound 170)

In a manner similar to Example 3, Step E, the reaction of 0.5 gram (0.00082 mole) of bis[4-(trifluoromethoxy)phenyl]{1-[(4-((1,3-dioxolan-2-yl)-3-methylphenyl)methyl](4-piperidyl)}methan-1-ol (Compound 169) and 6.2 mL of aqueous 30% hydrogen peroxide in 27 mL of methanol produced 0.5 gram of Compound 170 as a solid, melting point 165-168° C. The NMR spectrum was consistent with the proposed structure.

It is well known to one of ordinary skill in the art that compounds like the compounds of formula I of the present invention can contain optically active and racemic forms. It is also well known in the art that compounds like the compounds of formula I may contain stereoisomeric forms, tautomeric forms and/or exhibit polymorphism. It is to be understood that the present invention encompasses any racemic, optically active, polymorphic, tautomeric, or stereoisomeric form, or mixtures thereof. It should be noted that it is well known in the art how to prepare optically active forms, for example by resolution of a racemic mixture, or by synthesis from optically active intermediates.

The following table sets forth some additional examples of compounds of formula I useful in the present invention:

TABLE 1
Insecticidal Bis(substituted phenyl)-1-{[4-(saturated heterocyclyl-
substituted)phenylmethyl](4-piperidyl)}methane Derivatives

| | |
|---|---|
| | I |
| 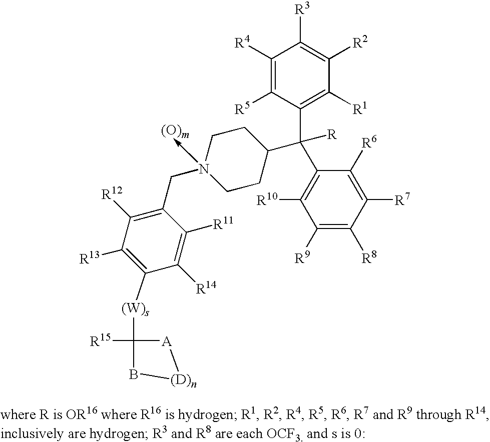 | |

| | |
|---|---|
| | I-1 |
| 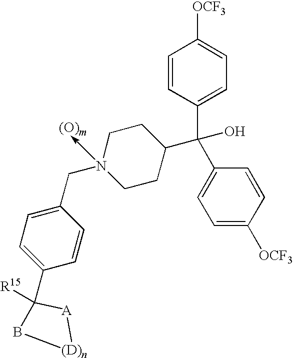 | |

| Cmpd.No. | m | 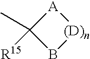 | Cmpd.No. | m | 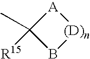 |
|---|---|---|---|---|---|
| 1 | 0 | 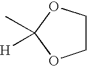 | 2 | 1 | 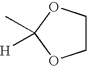 |
| 3 | 0 | 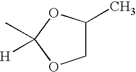 | 4 | 0 | 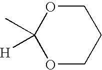 |
| 5 | 1 | 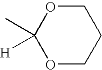 | 6 | 0 | 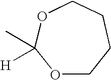 |
| 7 | 1 | 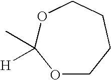 | 8 | 0 | 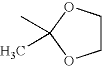 |
| 9 | 1 | 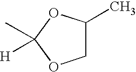 | 10 | 0 | 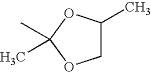 |
| 11 | 0 | 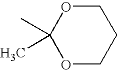 | 12 | 1 | 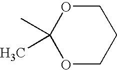 |
| 13 | 0 | 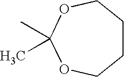 | 14 | 1 | 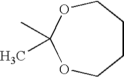 |
| 15 | 0 | 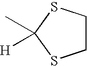 | 16 | 0 | 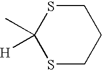 |
| 17 | 0 | 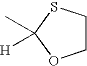 | 18 | 0 | 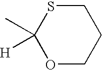 |
| 19 | 0 | 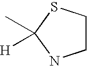 | 20 | 0 | 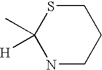 |
| 21 | 0 | 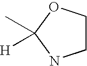 | 22 | 0 | 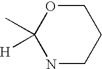 |
| 23 | 0 | 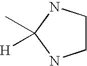 | 24 | 0 | 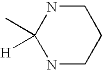 |
| 25 | 0 | 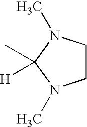 | 26 | 0 | 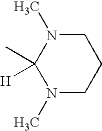 |
| 27 | 0 | 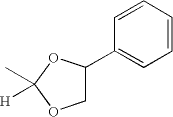 | 28 | 0 | 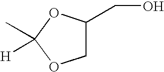 |
| 29 | 0 | 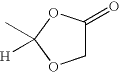 | 30 | 0 | 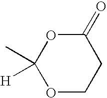 |
| 31 | 0 | 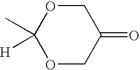 | 32 | 0 | 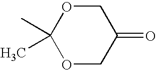 |
| 33 | 0 | 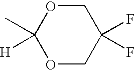 | 34 | 0 | 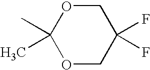 |
| 35 | 0 | 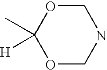 | 36 | 0 | 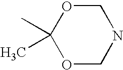 |
| 37 | 0 | 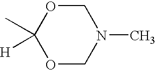 | 38 | 0 | 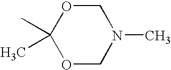 |
| 39 | 0 | 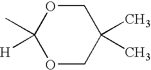 | 40 | 0 | 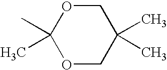 |
| 41 | 0 | 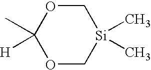 | 42 | 0 | 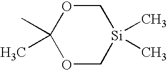 |
| 43 | 1 | 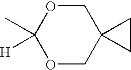 | 44 | 0 | 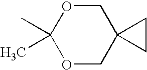 |
| 45 | 0 | 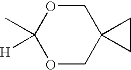 | 46 | 1 | 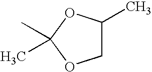 |
| 47 | 0 | 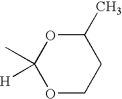 | 48 | 0 | 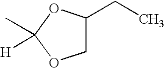 |
| 49 | 0 | 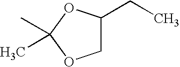 | 50 | 0 | 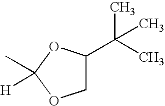 |
| 51 | 0 | 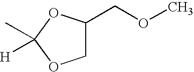 | 52 | 0 | 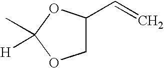 |
| 53 | 0 | 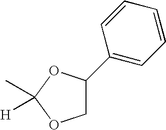 | 54 | 0 | 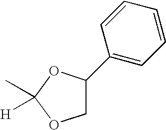 |
| 55 | 0 | 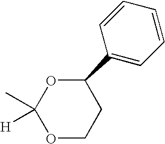 | 56 | 1 | 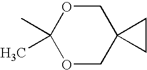 |
| 57 | 0 | 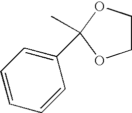 | 58 | 0 | 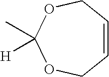 |
| 59 | 0 | 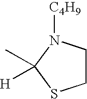 | 60 | 1 | 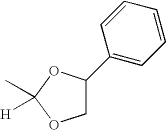 |
| 61 | 1 | 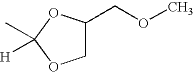 | 62 | 1 | 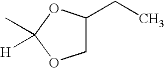 |
| 63 | 1 | 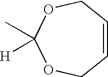 | 64 | 1 | 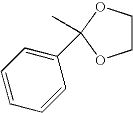 |
| 65 | 1 | 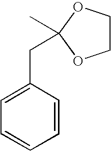 | 66 | 0 | 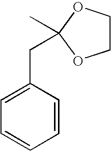 |
| 67 | 1 | 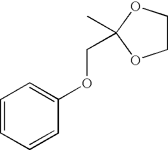 | 68 | 1 | 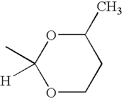 |
| 69 | 0 | 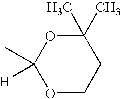 | 70 | 1 | 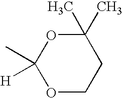 |
| 71 | 0 | 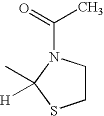 | 72 | 0 | 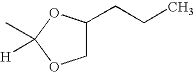 |
| 73 | 0 | 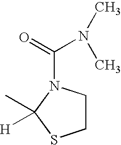 | 74 | 1 | 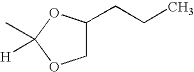 |
| 75 | 0 | 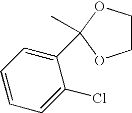 | 76 | 0 | 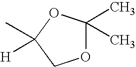 |
| 77 | 1 | 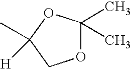 | 78 | 0 | 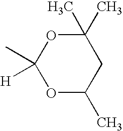 |
| 79 | 1 | 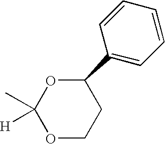 | 80 | 1 | 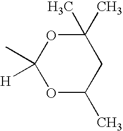 |
| 81 | 1 | 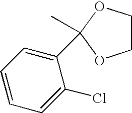 | 82 | 0 | 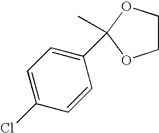 |
| 83 | 0 | 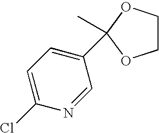 | 84 | 1 | 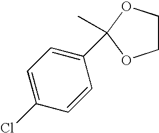 |
| 85 | 0 | 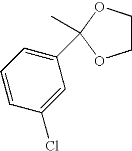 | 86 | 1 | 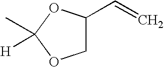 |
| 87 | 1 | 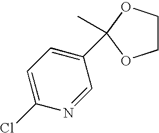 | 88 | 1 | 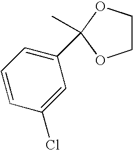 |
| 89 | 0 | 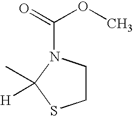 | 90 | 0 | 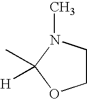 |
| 91 | 0 | 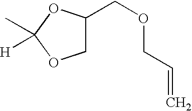 | 92 | 0 | 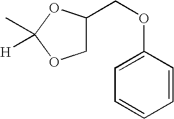 |
| 93 | 1 | 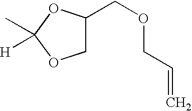 | 94 | 1 | 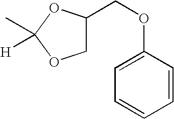 |
| 95 | 0 | 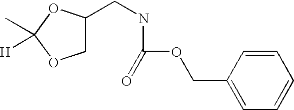 | 96 | 1 | 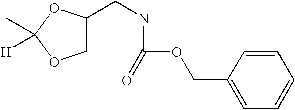 |
| 97 | 0 | 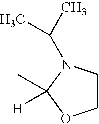 | 98 | 0 | 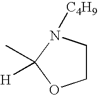 |
| 99 | 0 | 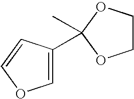 | 100 | 0 | 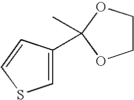 |
| 101 | 1 | 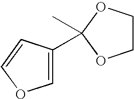 | 102 | 1 | 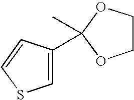 |
| 103 | 0 | 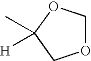 | 104 | 1 | 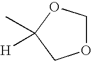 |
| 105 | 1 | 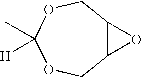 | 106 | 0 | 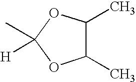 |
| 107 | 0 | 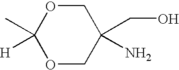 | 108 | 0 | 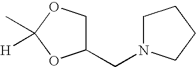 |
| 109 | 1 | 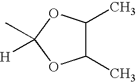 | 110 | 1 | 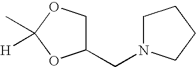 |
| 111 | 0 | 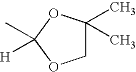 | 112 | 1 | 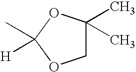 |
| 113 | 1 | 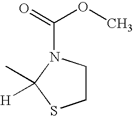 | 114 | 1 | 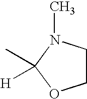 |
| 115 | 0 | 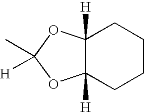 | 116 | 0 | 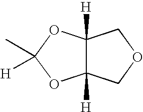 |
| 117 | 1 | 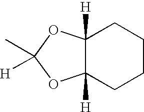 | 118 | 1 | 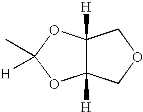 |
| 119 | 0 | 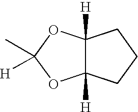 | 120 | 0 | 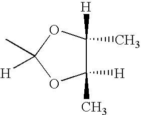 |
| 121 | 0 | 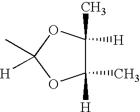 | 122 | 0 | 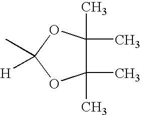 |
| 123 | 1 | 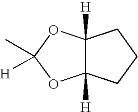 | 124 | 1 | 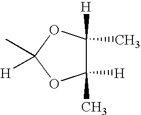 |
| 125 | 1 | 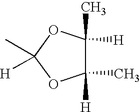 | 126 | 1 | 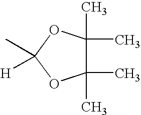 |
| 127 | 0 | 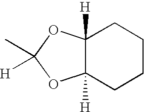 | 128 | 1 | 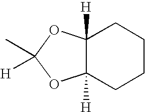 |

| | |
|---|---|
| | I-2 |
| 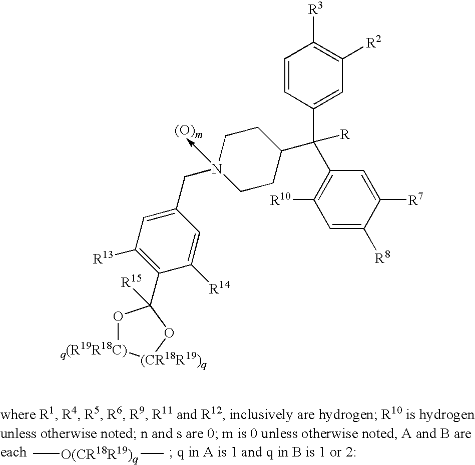 | |

| Cmpd.No. | R | R² | R³ | R⁷ | R⁸ | R¹³ | R¹⁴ | 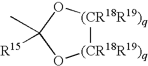 |
|---|---|---|---|---|---|---|---|---|
| 129 | H | H | OCF₃ | H | OCF₃ | H | H | 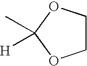 |
| 130 | H | H | OCF₃ | H | OCF₃ | H | H | 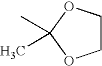 |
| 131 | F | H | OCF₃ | H | OCF₃ | H | H | 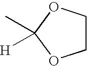 |
| 132 | F | H | OCF₃ | H | OCF₃ | H | H | 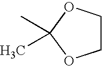 |
| 133 | NH₂ | H | OCF₃ | H | OCF₃ | H | H | 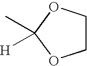 |
| 134 | NH₂ | H | OCF₃ | H | OCF₃ | H | H | 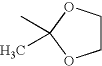 |
| 135 | NHC(═O)CH₃ | H | OCF₃ | H | OCF₃ | H | H | 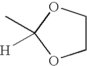 |
| 136 | NHC(═O)CH₃ | H | OCF₃ | H | OCF₃ | H | H | 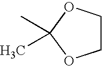 |
| 137 | NHC(═O)CH₂Cl | H | OCF₃ | H | OCF₃ | H | H | 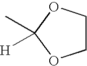 |
| 138 | NHC(═O)CH₂Cl | H | OCF₃ | H | OCF₃ | H | H | 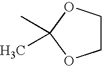 |
| 139 | NHC(═O)OCH₃ | H | OCF₃ | H | OCF₃ | H | H | 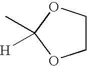 |
| 140 | NHC(═O)OCH₃ | H | OCF₃ | H | OCF₃ | H | H | 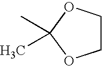 |
| 141 | NHC(═O)NHCH₃ | H | OCF₃ | H | OCF₃ | H | H | 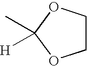 |
| 142 | NHC(═O)NHCH₃ | H | OCF₃ | H | OCF₃ | H | H | 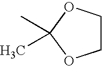 |
| 143 | OH | H | Cl | H | Cl | H | H | 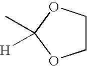 |
| 144 | OH | H | Cl | H | Cl | H | H | 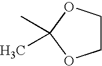 |
| 145 | OH | Cl | Cl | Cl | Cl | H | H | 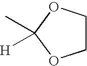 |
| 146 | OH | Cl | Cl | Cl | Cl | H | H | 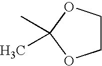 |
| 147 | OCH₃ | H | OCF₃ | H | OCF₃ | Cl | Cl | 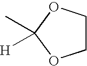 |
| 148 | OCH₃ | H | OCF₃ | H | OCF₃ | Cl | Cl | 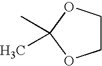 |
| 149 | H | H | OCF₃ | H | OCF₃ | H | H | 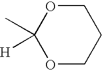 |
| 150 | H | H | OCF₃ | H | OCF₃ | H | H | 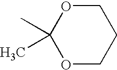 |
| 151 | F | H | OCF₃ | H | OCF₃ | H | H | 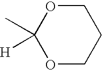 |
| 152 | F | H | OCF₃ | H | OCF₃ | H | H | 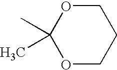 |
| 153 | NH₂ | H | OCF₃ | H | OCF₃ | H | H | 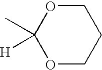 |
| 154 | NH₂ | H | OCF₃ | H | OCF₃ | H | H | 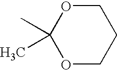 |
| 155 | NHC(═O)CH₃ | H | OCF₃ | H | OCF₃ | H | H | 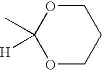 |
| 156 | NHC(═O)CH₃ | H | OCF₃ | H | OCF₃ | H | H | 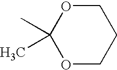 |
| 157 | NHC(═O)CH₂Cl | H | OCF₃ | H | OCF₃ | H | H | 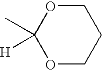 |
| 158 | NHC(═O)CH₂Cl | H | OCF₃ | H | OCF₃ | H | H | 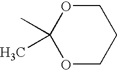 |
| 159 | NHC(═O)OCH₃ | H | OCF₃ | H | OCF₃ | H | H | 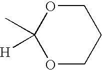 |
| 160 | NHC(═O)OCH₃ | H | OCF₃ | H | OCF₃ | H | H | 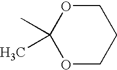 |
| 161 | NHC(═O)NHCH₃ | H | OCF₃ | H | OCF₃ | H | H | 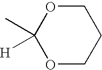 |
| 162 | NHC(═O)NHCH₃ | H | OCF₃ | H | OCF₃ | H | H | 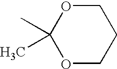 |
| 163 | OH | H | Cl | H | Cl | H | H | 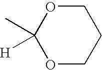 |
| 164 | OH | H | Cl | H | Cl | H | H | 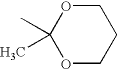 |
| 165 | OH | Cl | Cl | Cl | Cl | H | H | 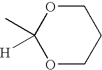 |
| 166 | OH | Cl | Cl | Cl | Cl | H | H | 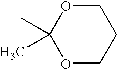 |
| 167 | OCH₃ | H | OCF₃ | H | OCF₃ | Cl | Cl | 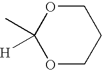 |
| 168 | OCH₃ | H | OCF₃ | H | OCF₃ | Cl | Cl | 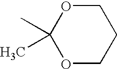 |
| 169 | OH | H | OCF₃ | H | OCF₃ | H | CH₃ | 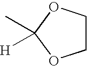 |
| 170* | OH | H | OCF₃ | H | OCF₃ | H | CH₃ | 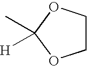 |
| 171 | OH | H | OCF₃ | H | OCF₃ | H | Cl | 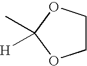 |
| 172 | OH | H | OCF₃ | H | OCF₃ | H | CF₃ | 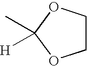 |
| 173 | OH | H | OCF₃ | H | OCF₃ | H | F | 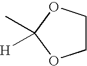 |
| 174** | OH | H | OCF₃ | H | H | H | H | 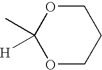 |
| 175* | OH | H | OCF₃ | H | OCF₃ | H | F | 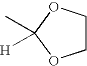 |
| 176 | OH | Cl | Cl | Cl | Cl | H | H | 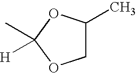 |
| 177* | OH | Cl | Cl | Cl | Cl | H | H | 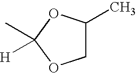 |
| 178 | H | H | OCF₃ | H | OCF₃ | H | H | 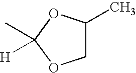 |
| 179 | OH | H | OCF₃ | H | OCF₃ | H | F | 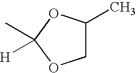 |
| 180* | OH | H | OCF₃ | H | OCF₃ | H | F | 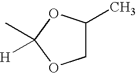 |
| 181* | OH | H | OCF₃ | H | OCF₃ | H | Cl | 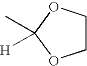 |
| 182* | OH | H | OCF₃ | H | OCF₃ | H | CF₃ | 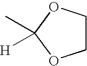 |

*m is 1
**R¹⁰ is OCF₃

| | |
|---|---|
| | I-3 |
| 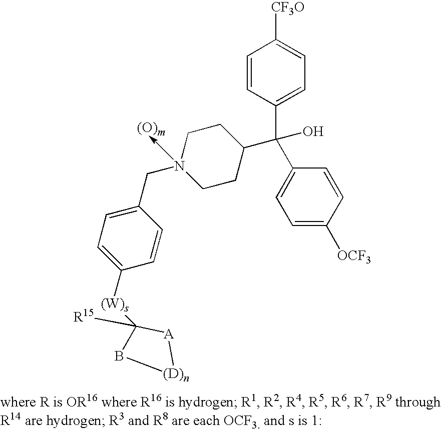 | |

| Cmpd.No. | m | W | 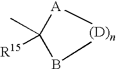 |
|---|---|---|---|
| 183 | 0 | —CH₂— | 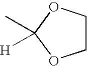 |
| 184 | 1 | —CH₂— | 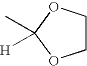 |
| 185 | 1 | —OCH₂— | 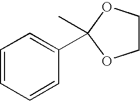 |
| 186 | 0 | —OCH₂— | 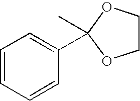 |
| 187 | 0 | —OCH₂— | 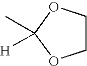 |
| 188 | 1 | —OCH₂— | 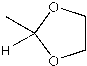 |
| 189 | 0 | —OCH₂— | 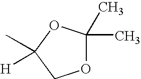 |
| 190 | 1 | —OCH₂— | 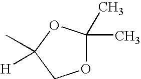 |
| 191 | 1 | —OCH₂— | 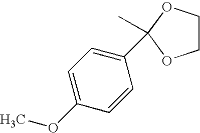 |
| 192 | 0 | —OCH₂— | 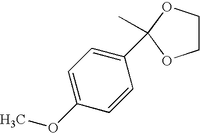 |
| 193 | 0 | —OCH₂— | 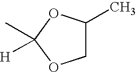 |
| 194 | 1 | —OCH₂— | 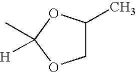 |
| 195 | 0 | —OCH₂— | 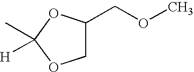 |
| 196 | 0 | —OCH₂— | 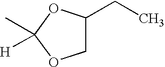 |
| 197 | 1 | —OCH₂— | 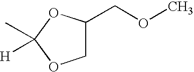 |
| 198 | 1 | —OCH₂— | 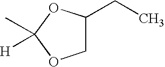 |
| 199 | 0 | —OCH₂— | 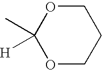 |
| 200 | 1 | —OCH₂— | 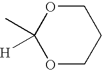 |

* R is hydrogen

The following table sets forth physical characterizing data for compounds of formula I of the present invention:

TABLE 2
Insecticidal Bis(substituted phenyl)-1-{[4-(saturated heterocyclyl-
substituted)phenylmethyl](4-piperidyl)}methane Derivatives
Characterizing Data

| | | Physical State | | | Physical State |
|---|---|---|---|---|---|
| Cmpd. | Emperical | or Melting | Cmpd. | Emperical | or Melting |
| No. | Formulae | Pt. ° C. | No. | Formulae | Pt. ° C. |
| 1 | C₃₀H₂₉F₆NO₅ | 51-55 | 2 | C₃₀H₂₉F₆NO₆ | 98-108 |
| 3 | C₃₁H₃₁F₆NO₅ | Glass | 4 | C₃₁H₃₁F₆NO₅ | 57-61 |
| 5 | C₃₁H₃₁F₆NO₆ | 92-102 | 6 | C₃₂H₃₃F₆NO₅ | Glass |
| 7 | C₃₂H₃₃F₆NO₆ | 178-181 | 8 | C₃₁H₃₁F₆NO₅ | 45-49 |
| 9 | C₃₁H₃₁F₆NO₆ | Solid | 11 | C₃₂H₃₃F₆NO₅ | 50-54 |
| 15 | C₃₀H₂₉F₆NO₃S₂ | 56-60 | 16 | C₃₁H₃₁F₆NO₃S₂ | 165-168 |
| 17 | C₃₀H₂₉F₆NO₄S | Glass | 18 | C₃₁H₃₁F₆NO₄S | Glass |
| 25 | C₃₂H₃₅F₆N₃O₃ | 56-62 | 39 | C₃₃H₃₅F₆NO₅ | 62-70 |
| 40 | C₃₄H₃₇F₆NO₅ | 52-56 | 45 | C₃₃H₃₃F₆NO₅ | 63-67 |
| 47 | C₃₂H₃₃F₆NO₅ | Oil | 48 | C₃₂H₃₃F₆NO₅ | 41-45 |
| 50 | C₃₄H₃₇F₆NO₅ | 53-57 | 51 | C₃₂H₃₃F₆NO₆ | 54-59 |
| 52 | C₃₂H₃₁F₆NO₅ | 41-44 | 53 | C₃₆H₃₃F₆NO₅ | Oil |
| 55 | C₃₇H₃₅F₆NO₅ | Oil | 57 | C₃₆H₃₃F₆NO₅ | 50-53 |
| 58 | C₃₂H₃₁F₆NO₅ | Glass | 59 | C₃₄H₃₈F₆N₂O₃S | Glass |
| 60 | C₃₆H₃₃F₆NO₆ | Solid | 61 | C₃₂H₃₃F₆NO₇ | Oil |
| 62 | C₃₂H₃₃F₆NO₆ | 182-184 | 63 | C₃₂H₃₁F₆NO₆ | 179-183 |
| 64 | C₃₆H₃₃F₆NO₆ | 60-65 | 65 | C₃₇H₃₅F₆NO₆ | 57-62 |
| 66 | C₃₇H₃₅F₆NO₅ | 50-56 | 67 | C₃₇H₃₅F₆NO₇ | 63-68 |
| 68 | C₃₂H₃₃F₆NO₆ | Solid | 69 | C₃₃H₃₅F₆NO₅ | Oil |
| 70 | C₃₃H₃₅F₆NO₆ | Solid | 71 | C₃₂H₃₂F₆N₂O₄S | Foam |
| 72 | C₃₃H₃₅F₆NO₅ | 42-48 | 73 | C₃₃H₃₅F₆N₃O₄S | Foam |
| 74 | C₃₃H₃₅F₆NO₆ | 108-112 | 75 | C₃₆H₃₂ClF₆NO₅ | Solid |
| 76 | C₃₂H₃₃F₆NO₅ | Semi Solid | 77 | C₃₂H₃₃F₆NO₆ | 55-59 |
| 78 | C₃₄H₃₇F₆NO₅ | Oil | 79 | C₃₇H₃₅F₆NO₆ | Solid |
| 80 | C₃₄H₃₇F₆NO₆ | Solid | 81 | C₃₆H₃₂ClF₆NO₆ | Solid |
| 82 | C₃₆H₃₂ClF₆NO₅ | Solid | 83 | C₃₅H₃₁ClF₆N₂O₅ | Solid |
| 84 | C₃₆H₃₂ClF₆NO₆ | Solid | 85 | C₃₆H₃₂ClF₆NO₅ | Solid |
| 86 | C₃₂H₃₁F₆NO₆ | Solid | 87 | C₃₅H₃₁ClF₆N₂O₆ | Solid |
| 88 | C₃₆H₃₂ClF₆NO₆ | Solid | 89 | C₃₂H₃₂F₆N₂O₅S | Foam |
| 90 | C₃₁H₃₂F₆N₂O₄ | Solid | 91 | C₃₄H₃₅F₆NO₆ | Glass |
| 92 | C₃₇H₃₅F₆NO₆ | Foam | 93 | C₃₄H₃₅F₆NO₇ | 90-95 |
| 94 | C₃₇H₃₅F₆NO₇ | 105-109 | 95 | C₃₉H₃₈F₆N₂O₇ | Foam |
| 96 | C₃₉H₃₈F₆N₂O₈ | Solid | 97 | C₃₃H₃₆F₆N₂O₄ | Solid |
| 98 | C₃₄H₃₈F₆N₂O₄ | Solid | 99 | C₃₄H₃₁F₆NO₆ | Solid |
| 100 | C₃₄H₃₁F₆NO₅S | Solid | 101 | C₃₄H₃₁F₆NO₇ | Solid |
| 102 | C₃₄H₃₁F₆NO₆S | Solid | 103 | C₃₀H₂₉F₆NO₅ | 48-52 |
| 104 | C₃₀H₂₉F₆NO₆ | 64-71 | 105 | C₃₂H₃₁F₆NO₇ | Solid |
| 106 | C₃₂H₃₃F₆NO₅ | 55-57 | 107 | C₃₂H₃₄F₆N₂O₆ | Solid |
| 108 | C₃₅H₃₈F₆N₂O₅ | Glass | 109 | C₃₂H₃₃F₆NO₆ | 185-187 |
| 110 | C₃₅H₃₈F₆N₂O₆ | Glass | 111 | C₃₂H₃₃F₆NO₅ | 49-51 |
| 112 | C₃₂H₃₃F₆NO₆ | 192-195 | 115 | C₃₄H₃₅F₆NO₅ | Glass |
| 116 | C₃₂H₃₁F₆NO₆ | Glass | 117 | C₃₄H₃₅F₆NO₆ | Solid |
| 118 | C₃₂H₃₁F₆NO₇ | Solid | 119 | C₃₃H₃₃F₆NO₅ | 74-78 |
| 120 | C₃₂H₃₃F₆NO₅ | Glass | 121 | C₃₂H₃₃F₆NO₅ | Glass |
| 122 | C₃₄H₃₇F₆NO₅ | Glass | 123 | C₃₃H₃₃F₆NO₆ | Solid |
| 124 | C₃₂H₃₃F₆NO₆ | Solid | 125 | C₃₂H₃₃F₆NO₆ | Glass |
| 126 | C₃₄H₃₇F₆NO₆ | 199-202 | 127 | C₃₄H₃₅F₆NO₅ | Solid |
| 128 | C₃₄H₃₅F₆NO₆ | Solid | 145 | C₂₈H₂₇Cl₄NO₃ | Oil |
| 169 | C₃₁H₃₁F₆NO₅ | 57-60 | 170 | C₃₁H₃₁F₆NO₆ | 165-168 |
| 171 | C₃₀H₂₈ClF₆NO₅ | 51-56 | 172 | C₃₁H₂₈F₉NO₅ | 50-55 |
| 173 | C₃₀H₂₈F₇NO₅ | Foam | 174 | C₃₁H₃₁F₆NO₅ | Oil |
| 175 | C₃₀H₂₈F₇NO₆ | Solid | 176 | C₂₉H₂₉Cl₄NO₃ | Oil |
| 177 | C₂₉H₂₉Cl₄NO₄ | Solid | 178 | C₃₁H₃₁F₆NO₄ | Oil |
| 179 | C₃₁H₃₀F₇NO₅ | Oil | 180 | C₃₁H₃₀F₇NO₆ | Solid |
| 181 | C₃₀H₂₈ClF₆NO₆ | 148-151 | 182 | C₃₁H₂₈F₉NO₆ | Solid |
| 183 | C₃₁H₃₁F₆NO₅ | 49-52 | 184 | C₃₁H₃₁F₆NO₆ | 59-63 |
| 185 | C₃₇H₃₅F₆NO₇ | 59-63 | 186 | C₃₇H₃₅F₆NO₆ | 48-53 |
| 187 | C₃₁H₃₁F₆NO₆ | Syrup | 188 | C₃₁H₃₁F₆NO₇ | 69-73 |
| 189 | C₃₃H₃₅F₆NO₆ | Syrup | 190 | C₃₃H₃₅F₆NO₇ | 96-107 |
| 191 | C₃₈H₃₇F₆NO₈ | 53-58 | 192 | C₃₈H₃₇F₆NO₇ | 45-50 |
| 193 | C₃₂H₃₃F₆NO₆ | Oil | 194 | C₃₂H₃₃F₆NO₇ | Solid |
| 195 | C₃₃H₃₅F₆NO₇ | Oil | 196 | C₃₃H₃₅F₆NO₆ | Oil |
| 197 | C₃₃H₃₅F₆NO₈ | Oil | 198 | C₃₃H₃₅F₆NO₇ | Solid |
| 199 | C₃₂H₃₃F₆NO₆ | Oil | 200 | C₃₂H₃₃F₆NO₇ | Solid |

Candidate insecticides were evaluated for activity against the tobacco budworm (Heliothis virescens [Fabricius]) in a surface-treated diet test.

In this test one mL of molten (65-70° C.) wheat germ-based artificial diet was pipetted into each well of a four by six (24 well) multi-well plate (ID# 430345-15.5 mm dia.×17.6 mm deep; Corning Costar Corp., One Alewife Center, Cambridge, Mass. 02140). The diet was allowed to cool to ambient temperature before treatment with candidate insecticide.

For a determination of insecticidal activity, solutions of the candidate insecticides were prepared for testing using a Packard 204DT Multiprobe® Robotic System (Packard Instrument Company, 800 Research Parkway, Meriden, Conn. 06450), in which the robot first diluted a standard 50 millimolar DMSO solution of candidate insecticide with a 1:1 water/acetone solution (V/V) in a ratio of 1:7 stock solution to water/acetone. The robot subsequently pipetted 40 microliters of the so-prepared solution onto the surface of the diet in each of three wells in the 24 multi-well plate. The process was repeated with solutions of seven other candidate insecticides. Once treated, the contents of the multi-well plate were allowed to dry, leaving 0.25 millimoles of candidate insecticide on the surface of the diet, or a concentration of 0.25 millimolar. Appropriate untreated controls containing only DMSO on the diet surface were also included in this test.

For evaluations of the insecticidal activity of a candidate insecticide at varying rates of application, the test was established as described above using sub-multiples of the standard 50 millimolar DMSO solution of candidate insecticide. For example, the standard 50 millimolar solution was diluted by the robot with DMSO to give 5, 0.5, 0.05, 0.005, 0.0005 millimolar, or more dilute solutions of the candidate insecticide. In these evaluations there were six replicates of each rate of application placed on the surface of the diet in the 24 multi-well plate, for a total of four rates of application of candidate insecticide in each plate.

In each well of the test plate was placed one second instar tobacco budworm larvae, each weighing approximately five milligrams. After the larvae were placed in each well, the plate was sealed with clear polyfilm adhesive tape. The tape over each well was perforated to ensure an adequate air supply. The plates were then held in a growth chamber at 25° C. and 60% relative humidity for five days (light 14 hours/day).

After the five-day exposure period insecticidal activity for each rate of application of candidate insecticide was assessed as percent inhibition of insect weight relative to the weight of insects from untreated controls, and percent mortality when compared to the total number of insects infested.

Insecticidal activity data at selected rates of application from this test are provided in Table 3. The test compounds of formula I are identified by numbers that correspond to those in Table 1.

TABLE 3
Insecticidal Activity of Certain Bis(substituted phenyl)-1-{[4-(saturated
heterocyclyl-substituted)phenylmethyl](4-piperidyl)}methane
Derivatives When Applied to the Surface of the Diet of Tobacco
Budworm (Heliothis virescens [Fabricius])

| | | Percent | | | Percent |
|---|---|---|---|---|---|
| Cmpd. | Percent | Growth | Cmpd. | Percent | Growth |
| No | Mortality | Inhibition | No | Mortality | Inhibition |
| 1 | 100 | 100 | 2 | 100 | 100 |
| 3 | 100 | 100 | 4 | 100 | 100 |
| 5 | 100 | 100 | 6 | 100 | 100 |
| 7 | 100 | 100 | 8 | 100 | 100 |
| 9 | 100 | 100 | 11 | 100 | 100 |
| 15 | 100 | 100 | 16 | 100 | 100 |
| 17 | 100 | 100 | 18 | 100 | 100 |
| 25 | 100 | 100 | 39 | 100 | 100 |
| 40 | 100 | 100 | 45 | 100 | 100 |
| 47 | 67 | 100 | 48 | 100 | 100 |
| 50 | 100 | 100 | 51 | 100 | 100 |
| 52 | 100 | 100 | 53 | 100 | 100 |
| 55 | 100 | 100 | 57 | 100 | 100 |
| 58 | 100 | 100 | 59 | 100 | 100 |
| 60 | 100 | 100 | 61 | 100 | 100 |
| 62 | 100 | 100 | 63 | 100 | 100 |
| 64 | 100 | 100 | 65 | 67 | 100 |
| 66 | 67 | 100 | 67 | 100 | 100 |
| 68 | 100 | 100 | 69 | 100 | 100 |
| 70 | 100 | 100 | 71 | 33 | 100 |
| 72 | 100 | 100 | 73 | 100 | 100 |
| 74 | 100 | 100 | 75 | 100 | 100 |
| 76 | 100 | 100 | 77 | 100 | 100 |
| 78 | 100 | 100 | 79 | 100 | 100 |
| 80 | 100 | 100 | 81 | 100 | 100 |
| 82 | 100 | 100 | 83 | 0 | 31 |
| 84 | 100 | 100 | 85 | 100 | 100 |
| 86 | 100 | 100 | 87 | 17 | 79 |
| 88 | 100 | 100 | 89 | 100 | 100 |
| 90 | 100 | 100 | 91 | 100 | 100 |
| 92 | 100 | 100 | 93 | 100 | 100 |
| 94 | 100 | 100 | 95 | 100 | 100 |
| 96 | 100 | 100 | 97 | 50 | 100 |
| 98 | 100 | 100 | 99 | 100 | 100 |
| 100 | 0 | 100 | 101 | 100 | 100 |
| 102 | 100 | 100 | 103 | 100 | 100 |
| 104 | 100 | 100 | 105 | 100 | 100 |
| 106 | 100 | 100 | 107 | 100 | 100 |
| 108 | 33 | 97 | 109 | 100 | 100 |
| 110 | 100 | 100 | 111 | 100 | 100 |
| 112 | 100 | 100 | 115 | 100 | 100 |
| 116 | 100 | 100 | 117 | 100 | 100 |
| 118 | 100 | 100 | 119 | 100 | 100 |
| 120 | 100 | 100 | 121 | 100 | 100 |
| 122 | 100 | 100 | 123 | 100 | 100 |
| 124 | 100 | 100 | 125 | 100 | 100 |
| 126 | 100 | 100 | 127 | 100 | 100 |
| 128 | 100 | 100 | 145 | 100 | 100 |
| 169 | 100 | 100 | 170 | 100 | 100 |
| 171 | 100 | 100 | 172 | 100 | 100 |
| 173 | 100 | 100 | 174 | 100 | 100 |
| 175 | 100 | 100 | 176 | 100 | 100 |
| 177 | 100 | 100 | 178 | 100 | 100 |
| 179 | 100 | 100 | 180 | 100 | 100 |
| 181 | 100 | 100 | 182 | 100 | 100 |
| 183 | 100 | 100 | 184 | 100 | 100 |
| 185 | 100 | 100 | 186 | 100 | 100 |
| 187 | 100 | 100 | 188 | 100 | 100 |
| 189 | 100 | 100 | 190 | 100 | 100 |
| 191 | 100 | 100 | 192 | 100 | 100 |
| 193 | 100 | 100 | 194 | 100 | 100 |
| 195 | 100 | 100 | 196 | 100 | 100 |
| 197 | 100 | 100 | 198 | 100 | 100 |
| 199 | 100 | 100 | 200 | 100 | 100 |

Concentration of the candidate insecticide on the surface of the diet is 0.25 millimolar

While this invention has been described with an emphasis upon preferred embodiments, it will be understood by those of ordinary skill in the art that variations of the preferred embodiments may be used and that it is intended that the invention may be practiced otherwise than as specifically described herein. Accordingly, this invention includes all modifications encompassed within the spirit and scope of the invention as defined by the following claims.

What is claimed is:

1. A compound of formula I: 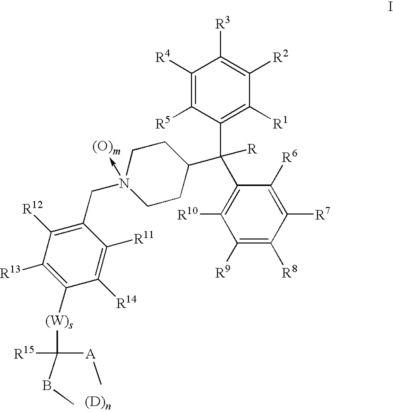
wherein
R is selected from hydrogen, OR¹⁶, SR¹⁶, halogen, cyano, alkyl, haloalkyl, alkylsilyl, alkoxysilyl, NR¹⁶R¹⁷, C(═O)R¹⁶, NHC(═O)R¹⁶, NHC(═O)OR¹⁶, NHC(═O)NHR¹⁶ and NHC(═S)NHR¹⁶; where R¹⁶ and R¹⁷ are independently selected from hydrogen, alkyl, haloalkyl, hydroxyalkyl, alkoxyalkyl, alkylsilyl, alkoxysilyl, aryl, arylalkyl and heteroaryl;
R¹ through R¹⁰, inclusively, are independently selected from hydrogen, halogen, alkyl, haloalkyl, alkoxy, haloalkoxy, S(O)ₚ-alkyl, S(O)ₚ-haloalkyl, pentahalothio and nitro; where p is an integer selected from 0, 1 or 2;
and
R¹ and R², R² and R³, R⁶ and R⁷, and R⁷ and R⁸ may be taken together with —OC(halogen)₂O— to form benzo-fused rings;
m is an integer selected from 0 or 1;
s is an integer selected from 0 or 1;
W is selected from —CR³⁰R³¹— or —OCR³OR³″—; where R³⁰ and R³¹ are independently selected from hydrogen or alkyl;
R¹¹ through R¹⁴, inclusively, are independently selected from hydrogen, halogen, alkyl, haloalkyl, alkoxyalkyl, OR¹⁶, S(O)ₚR¹⁶, NR¹⁶R¹⁷ and aryl; where p, R¹⁶ and R¹⁷ are previously described;
R¹⁵ is selected from hydrogen, alkyl, cycloalkyl, alkenyl, alkynyl, haloalkyl, hydroxyalkyl, alkoxyalkyl, OR¹⁶, SR¹⁶, aryl, arylalkyl, aryloxyalkyl and heteroaryl; where R¹⁶ is previously described;
n is an integer selected from 0 or 1;
and,
when n is 0 or 1;
A and B are independently selected from —O(C═O)—, —C(═O)O—, —O(CR¹⁸R¹⁹)_{q}—, —(CR¹⁸R¹⁹)_{q}O—, S(O)ₚ(CR¹⁸R¹⁹)_{q}—, —(CR¹⁸R¹⁹)_{q}S(O)ₚ—, —(CR¹⁸R¹⁹)_{q}NR²⁰— and —NR²⁰(CR¹⁸R¹⁹)_{q}—, where p is previously described, and q is an integer selected from 1 to 7, provided that the sum of q is 8 or less; R¹⁸ and R¹⁹ are independently selected from hydrogen, alkyl, alkenyl, alkynyl, haloalkyl, hydroxyalkyl, alkoxyalkyl, alkenyloxyalkyl, aryl, aryloxyalkyl, arylalkoxycarbonylaminoalkyl, heterocyclalkyl and heteroaryl; and R²⁰ is selected from hydrogen, alkyl, haloalkyl, hydroxyalkyl, alkoxyalkyl, acyl, alkylcarbonyl, alkoxycarbonyl, aminocarbonyl, alkylaminocarbonyl, dialkylaminocarbonyl, aryl, arylalkyl, silyl and phosphoryl; and when n is 1;
D is selected from —O—, —S—, —NR²⁰—, —C═NOR²⁰—, —C(halogen)₂-, —CR²¹R²²—, C(R²¹)(OR²²)—, —C(OR²¹)(OR²²)—, SiR²¹R²²—, —Si(OR²¹)(OR²²)—, P(═O)ᵣR²¹, P(═O)ᵣ(OR²¹), BR²¹, B(OR²¹), C(═O), C═CR²³R²⁴, —CR²³═CR²⁴— and 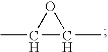
where R²⁰ is previously described; R²¹ and R²² are independently selected from hydrogen, alkyl, alkenyl, alkynyl, haloalkyl, hydroxyalkyl, alkoxyalkyl, amino, aryl and heteroaryl, and where R²¹ and R²² may be taken together to form a spiro ring; R²³ and R²⁴ are independently selected from hydrogen, halogen, alkyl, haloalkyl, hydroxyalkyl, alkoxyalkyl, alkylsilyl, alkoxysilyl, aryl, arylalkyl and heteroaryl; and r is an integer selected from 0 or 1;
or
when n is 1;
A and B are independently selected from selected from —O—, —S(O)ₚ—, —O(CR¹⁸R¹⁹)_{q}—, —(CR¹⁸R¹⁹)_{q}O—, —NR²⁰— and —(CR²¹R²²)ₜ— e p, q, R¹⁸, R¹⁹, R²⁰, and R²² are previously described; and t is an integer selected from 1, 2, 3, or 4;
and,
D is a phenylene moiety 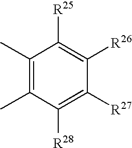
which,
when taken together with A and B, forms a benzo-fused ring, where R²⁵ through R²⁸ are independently selected from hydrogen, halogen, alkyl, haloalkyl, hydroxyalkyl, alkoxy, alkoxyalkoxy, alkylthio, thioalkyl, cyano, alkoxycarbonyl, aryl, aryloxy, heteroaryl and heteroaryloxy;
or
when n is 1;
A and B are independently selected from selected from —O—, —S(O)ₚ—, —O(CR¹⁸R¹⁹)_{q}—, (CR¹⁸R¹⁹)_{q}O—, —NR²⁰— and —(CR²¹R²²)ₜ—, where p, q, R¹⁸, R¹⁹, R²⁰, R²¹ and R²² are previously described; and t is an integer selected from 1, 2, 3, or 4;
and,
D is a cyclic moiety selected from: 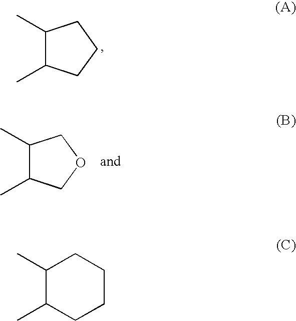
which,
when taken together with A and B, form a fused ring;
and agriculturally acceptable salts thereof;
provided that when R is OR¹⁶ where R¹⁶ is hydrogen; m is 0; R¹¹ through R¹⁵, inclusively are hydrogen; A and B are each —O(CR¹⁸R¹⁹)_{q}— where R¹⁸ and R¹⁹ are each hydrogen and q is 1; and n is 0, and i) when R¹, R², R⁴R⁵, R⁷, R⁸, R⁹ and R¹⁰ are hydrogen and R³ is trifluoromethoxy, then R⁶ is other than trifluoromethoxy; or ii) when R¹, R², R⁴R⁵, R⁶, R⁷, R⁸ and R⁹ are hydrogen and R³ is trifluoromethoxy, then R¹⁰ is other than trifluoromethoxy; or iii) when R², R³, R⁴R⁵, R⁶, R⁷, R⁹ and R¹⁰ are hydrogen and R⁸ is trifluoromethoxy, then R¹ is other than trifluoromethoxy, or iv) when R¹, R², R³R⁴, R⁶, R⁷, R⁹ and R¹⁰ are hydrogen and R⁸ is trifluoromethoxy, then R⁵ is other than trifluoromethoxy;
and provided that when A and B are independently selected from —C(═O)O—, —(CR¹⁸R¹⁹)_{q}O— and —(CR¹⁸R¹⁹)_{q}S(O)ₚ—, then D is not selected from —O— or —S—.

2. A compound of claim 1, wherein R is selected from hydrogen, fluorine, OR¹⁶ and NR¹⁶R¹⁷, where R¹⁶ and R¹⁷ are hydrogen; i) R¹, R², R⁴, R⁵, R⁶, R⁷, R⁹ and R¹⁰ are each hydrogen, and R³ and R⁸ are each trifluoromethyl or trifluoromethoxy; or ii) R¹, R⁴, R⁵, R⁶, R⁹ and R¹⁰ are each hydrogen, and R², R³, R⁷ and R⁸ are independently selected from fluorine or chlorine; R¹¹ through R¹⁴, inclusively, are independently selected from hydrogen, halogen, alkyl and alkoxy; R¹⁵ is hydrogen or alkyl; n is 0; A and B are independently selected from —O(CR¹⁸R¹⁹)_{q}—, —S(O)ₚ(CR¹⁸R¹⁹)_{q}— and —NR²⁰(CR¹⁸R¹⁹)_{q}—, where p is 0, and q is an integer selected from 1 to 3.

3. A compound of claim 2, wherein R is OR¹⁶, where R¹⁶ is hydrogen; R¹, R², R⁴, R⁵, R⁶, R⁷, R⁹ and R¹⁰ are each hydrogen, and R³ and R⁸ are each trifluoromethyl or trifluoromethoxy; R¹¹ through R¹³, inclusively, are each hydrogen; R¹⁴ is hydrogen, halogen or alkyl, q is an integer selected from 1 to 2; R¹⁵, is hydrogen; and R¹⁸ and R¹⁹ are hydrogen or alkyl.

4. A composition comprising an insecticidally effective amount of a compound of claim 1 and at least one agriculturally acceptable extender or adjuvant.

5. The insecticidal composition of claim 4, further comprising one or more second compounds selected from the group consisting of pesticides, plant growth regulators, fertilizers and soil conditioners.

6. A method of controlling insects, comprising applying an insecticidally effective amount of a composition of claim 4 to a locus where insects are present or are expected to be present.

7. A method of controlling insects, comprising applying an insecticidally effective amount of a composition of claim 5 to a locus where insects are present or are expected to be present.
